(19) United States
(12) Patent Application Publication
(10) Pub. No.: US 2002/0197331 A1
(43) Pub. Date: Dec. 26, 2002

(54) Chromium/biotin treatment of dyslipidemia and diet-induced post prandial hyperglycemia

(76) Inventors: James R. Komorowski, Trumbull, CT (US); Jon De La Harpe, New York, NY (US); Danielle Greenberg, Waccabuc, NY (US); Vijaya Juturu, Dobbs Ferry, NY (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/090,038

(22) Filed: Feb. 27, 2002

Related U.S. Application Data
(60) Provisional application No. 60/271,881, filed on Feb. 27, 2001.

(51) Int. Cl. A61K 35/84; A61K 33/24; A61K 31/555; A61K 31/4188

(52) U.S. Cl. 424/655; 424/195.15; 514/184; 514/393

(57) ABSTRACT

A method for treating dyslipidemia and/or post prandial hyperglycemia by administering a combination of a chromium complex and biotin to an individual in need thereof is disclosed. The two compounds are administered orally or parenterally in daily dosages which provide between 25 μg and 1,000 μg of chromium and between 25 μg and 20 mg biotin. A method for reducing the glycemic index of food is similarly provided.

### CROSS REFERENCE TO RELATED APPLICATIONS

The present application claims priority to Provisional Application No. 60/271,881 entitled CHROMIUM/BIOTIN TREATMENT OF HYPERCHOLESTEROLEMIA, filed on Feb. 27, 2001. The subject matter of the aforementioned application is hereby incorporated by reference in its entirety.

### BACKGROUND OF THE INVENTION

1. Field of the Invention

The present invention relates to the improvement of blood cholesterol and triglyceride levels. More specifically, the invention relates to methods of lowering LDL cholesterol, increasing HDL cholesterol, and decreasing triglyceride levels in the blood by administering doses of chromic picolinate and biotin. Additionally, the present invention relates to methods and compositions for reducing post prandial hyperglycemia and for lowering the glycemic index of foods.

2. Description of the Related Art

Dyslipidemia

Dyslipidemias are disorders of lipoprotein metabolism, including lipoprotein overproduction or deficiency. These disorders may be manifested by elevation of the serum total cholesterol, low-density lipoprotein (LDL) cholesterol and triglyceride concentrations, and a decrease in the high-density lipoprotein (HDL) cholesterol concentration. Each year, millions of human beings suffer from the sequelae of hypercholesterolemia. Examples of the afflictions include hypertension, coronary artery disease, congestive heart failure, peripheral vascular disease, aneurysms, and death due at least in part to these conditions. Elevated blood cholesterol is one of the major modifiable risk factors for coronary heart disease (CHD), the leading cause of death in the United States. Kannel, W. B. et al. Declining Cardiovascular Mortality. Circulation 70:331-336 (1984). CHD is responsible for roughly 490,000 deaths each year. National Center for Health Statistics. Annual summary of births, marriages, divorces, and deaths: United States, 1993. Monthly vital statistics report; vol 42 no 13. Public Health Service, 1994. Nonfatal myocardial infarction (MI) and angina are similarly a source of substantial morbidity. Secondary physiological effects of hypercholesterolemia include cerebral strokes, compromised liver function, renal artery blockage, senility, male impotence, and arteriosclerotic aneurysms. The risk of such diseases can be reduced by increasing the level of HDL cholesterol in the blood and/or decreasing the level of LDL cholesterol in the blood.

Renewed emphasis has been placed on lowering blood cholesterol, particularly the Low Density Lipoprotein (LDL) faction, the major reservoir of cholesterol in blood plasma. Many attempts have been made to lower the blood cholesterol level in individuals through dietary management, behavior modification, exercise, and drug therapy aimed at reducing or controlling the blood cholesterol levels.

The first recommendation in treating hypercholesterolemia is generally dietary intervention, whereby lipid intake has been restricted. Dr. Dean Ornish et. al. “Can Lifestyle Changes Reverse Coronary Heart Disease,” The Lancet. vol. 336 (1990) and his ongoing program for reversing heart disease, has shown that complete elimination of dietary cholesterol and limiting fat content to less than ten percent of the daily caloric intake can effect a four percent regression of atherosclerotic plaque after five years when combined with stress management and aerobic exercise. This strict vegetarian diet (free of meat, fish, chicken, vegetable oils and all dairy fat products) is unrealistic for most individuals.

Dietary supplements have offered some promise in the fight against hypercholesterolemia. For example, brans, psylliums, guar gum, lecithins, whey, red wines, fish oils and ginseng root extract have been reported to reduce high blood cholesterol or its consequences. The mechanisms are varied and include cholesterol sequestering, chelating, entrapment, and oxidation inhibition. Such regimens generally affect only less than ten percent reduction in blood cholesterol. None of these dietary interventions have been shown to arrest or cure atherosclerosis or other high blood cholesterol associated diseases.

The most severe dietary intervention has been reported in U.S. Pat. No. 5,032,608 where it describes intravenous feeding of a mixture of biologically active levorotatory amino acids which replace all other feedings except water. A functionally equivalent fat free dietary composition is described in U.S. Pat. No. 5,106,836 which discloses a method for preparing enteral food products with a modified fat free total parenteral nutritional amino acid formulation. The prepared foods, when digested and absorbed, deliver an amino acid profile into the blood having hypocholesterolemic properties. It is reported that a significant reduction in total plasma cholesterol and regression of atherosclerosis is shown if the diet is maintained.

Other attempts to lower serum cholesterol levels have been directed to the development of various pharmaceutical preparations. For example, a bacterial cell product has been reported in U.S. Pat. No. 4,797,278 to lower blood cholesterol and/or triglyceride levels by having the ability to adhere to intestinal epithelial cells of the intestine thereby optimizing the conditions for colonization of beneficial bacteria in the appropriate areas in the intestine.

U.S. Pat. No. 5,114,963 describes a method of reducing atherosclerotic disease by reducing serum levels of lipoprotein by the administration of N,S-diacryl-L-cystein. Methods of treating myocardial damage have also centered on the intravenous injection of compounds in combination with fibrinolytic enzymes to digest or dissolve blood thrombosis, lysing fibrin clots and re-establishing and maintaining perfusion of ischemic tissue. This use of enzymes, whose amelioration of the consequence of atherosclerosis is described in U.S. Pat. No. 5,028,599, does not lower blood cholesterol concentration.

In view of the above failures of dietary, enzyme therapy, and lifestyle interventions, other means have been sought to lower blood cholesterol, reverse arterial plaque deposits, and otherwise mitigate the effects of high blood cholesterol on other tissue. The lowering of cholesterol with hypocholesterolemic drugs, cholesterol anti-absorption drugs (e.g. melinamide, thioesters, substituted urea and thiourea), certain cyclodextrins that act as substitute apoproteins cholesterol carriers, and cholesterol biosynthesis limiting drugs to reduce the risk of coronary heart disease is supported by convincing evidence to have a causal association with the lowering of blood cholesterol levels. These drugs and their adverse side effects such as liver impairment are well described in the Physicians' Desk Reference Medical Economics Company Oradell, N.J.

None of the above drugs alone or in combination, have been shown to significantly lower blood cholesterol and reduce atherosclerosis plaque without requiring concurrent severe restriction on ingestion of lipids. To date, there remains a dearth of effective and safe drugs which would prevent and treat high cholesterol without any adverse side-effects. Therefore, an object of the present invention is to provide a safe and effective treatment for diseases associated with high cholesterol levels without the associated side effects and severe life style restrictions.

Post prandial hyperglycemia

Recent epidemiological studies indicate that the glycemic index (“GI”) of the diet may be the most important dietary factor in preventing type 2 diabetes. The GI is an established, physiologically based method used to classify foods according to their blood glucose-raising potential. See Joint FAO/WHO Expert Consultation, Apr. 14-18, 1997. Specifically, the glycemic index ranks foods on how they affect our blood sugar levels by measuring the increase in blood sugar after two or three hours following the consumption of food. The index compares the level of glycemia after equal carbohydrate portions of foods and ranks them relative to a standard (usually glucose or white bread).

Over the past two decades, the GI concept has been subjected to extensive research confirming its reproducibility, application to mixed meals, and clinical usefulness in the treatment of diabetes and hyperlipidemia. Wolever, T. M. S. et. al. Am J Clin Nutr 54:846-54 (1991); Brand-Miller, J. Am J Clin Nutr 59(suppl):747S-52S (1994). Long-term studies in animal models show that high-GI starch increases fasting insulin levels and promotes insulin resistance, in comparison with identical diets based on low-GI starch. Byrnes, S. et al., J Nutr 125:1430-7 (1995); Higgins, J. A. et al. J Nutr 126:596-602 (1996). In rats, high-GI diets promote faster weight gain, higher body fat levels, higher adipocyte volume, and hypertriglyceridemia—that is, all of the components of the insulin resistance or “metabolic” syndrome. See, eg. Pawlak, D. B. et al. Proc Nutr Soc Aust 21:143 (1997); Lerer-Metzger, M. et al. Br J Nutr 75; 723-32 (1996).

In subjects with type 1 and type 2 diabetes, low-GI diets, in comparison with high-GI diets of similar nutrient composition, lead to improvements in glucose and lipid metabolism. In eight well-designed long-term studies using a cross-over design, the low-GI diet reduced glycosylated proteins by an average of almost 14% over periods ranging from 2 to 12 weeks. Wolever, T. M. S. et al. Diabetes Care 15:562-4 (1992); Fontvieille, A. M. et al. Diabetic Medicine 9:444-50 (1992). Although these results have been criticized as only modest, they are higher in magnitude than improvements induced by oral hypoglycemic drugs. The improvement in glycosylated proteins with low-GI diets contrasts with the lack of change seen with high-MUFA (“MUFA”=Monounsaturated Fatty Acids) diets in diabetes. Wolever, T. M. S. Nutrition Today, 34:73-7 (1999).

Two large-scale prospective studies, one in female nurses and one in male health professionals, showed that diets with a high glycemic load (GI×carbohydrate content) increase the risk of developing type 2 diabetes after controlling for known risk factors such as age and body mass index. Salmeron, J. et al. JAMA 277:472-477 (1997); Salmeron, J. et al. Diabetes Care 20:545-550 (1997). The only other dietary factor that increased risk was lack of cereal fiber. Importantly, the total carbohydrate and refined sugar content, and the amount and type of fat, were not found to be independent risk factors in these studies. A similar picture has emerged with acute coronary heart disease in the Nurses' study. Liu, S. et al. FASEB J 124:A260 (abstr 1517) (1998). The underlying mechanism postulated by these authors is the demand for insulin generated by high-GI foods. Because hyperinsulinemia is linked with all of the facets of the “metabolic syndrome” (insulin resistance, hyperlipidemia, hypertension, and visceral obesity), the GI of foods eventually may be linked with all so-called diseases of affluence.

In healthy people as well as those with type 2 diabetes, high-carbohydrate diets (i.e., >50% energy) have been shown to worsen aspects of the blood lipid profile. See Garg, A. et al. Diabetes 41;1278-1285 (1992); Mensink, R. P. et al. Metab 38:172-178 (1989). Individuals with insulin resistance are more susceptible to these adverse effects. However, the effect of high-carbohydrate diets is almost certainly linked to the rate of absorption of the carbohydrate, because strategies that slow down digestion and absorption (high soluble fiber, low GI, α-glucosidase therapy) improve these parameters. Albrink, M. J. et al., Am J Clin Nutr 32:1486-1491 (1979). The concerns with usual (i.e., high-GI) high-carbohydrate diets have led some experts to recommend high intake of monounsaturated and polyunsaturated oils in place of carbohydrate, (Storlien, L. H. et al.,. Diabetologia 39:621-31(1996)) but high-fat, energy-dense diets of any sort are prone to over consumption. High-carbohydrates foods (even energy-dense versions) can only ever have half the energy density of high-fat foods. A primary goal in the management of diabetes is to prevent its long-term complications through the attainment of tight glycemic control, a complex and incompletely understood metabolic process that involves the interaction of the pancreas, insulin-responsive peripheral tissues, and the liver in regulating fasting blood glucose (FBG) and postprandial glucose (PPG) levels. Whereas much of the clinical management of type 2 diabetes has focused on FBG by measurement of blood glucose levels and glycated hemoglobin levels, mounting evidence has formed strong associations between PPG, the temporally immediate physiological management of a glucose load, and diabetes progression, management, and complications. Application of the research may enable health care systems and providers to more closely mimic a normal glycemic response in individuals with type 2 diabetes, leading to improved clinical outcomes and cost control.

Early identification of elevated post prandial blood glucose levels is an important step in predicting the onset of microvascular and macrovascular complications that can progress to full symptomatic diabetes. A growing body of evidence indicates that measurements of post prandial glucose levels, in combination with glycosylated hemoglobin, are a more accurate predictor of metabolic abnormality than fasting or pre-prandial glucose levels for individuals with type 2 diabetes.

The Role of Chromium

Dietary supplementation of chromium to normal individuals has been reported to lead to improvements in glucose tolerance, serum lipid concentrations, including high-density lipoprotein cholesterol, insulin and insulin binding (Anderson, Clin. Psychol. Biochem. 4:31-41, 1986). Supplemental chromium in the trivalent form, e.g. chromic chloride, is associated with improvements of risk factors associated with adult-onset (Type 2) diabetes and cardiovascular disease.

Chromium is a nutritionally essential trace element. The essentiality of chromium in the diet was established in 1959 by Schwartz, as cited in Present Knowledge in Nutrition, page 571, fifth edition (1984, the Nutrition Foundation, Washington, D.C.). Chromium depletion is characterized by the disturbance of glucose, lipid and protein metabolism and by a shortened lifespan. Chromium is essential for optimal insulin activity in all known insulin-dependent systems (Boyle et al., Southern Med. J. 70:1449-1453, 1977). Insufficient dietary chromium has been linked to both maturity-onset diabetes and to cardiovascular disease.

The principal energy sources for the body are glucose and fatty acids. Chromium depletion results in biologically ineffective insulin and compromised glucose metabolism. Under these conditions, the body must rely primarily on lipid metabolism to meet its energy requirements, resulting in the production of excessive amounts of acetyl-CoA and ketone bodies. Some of the documented acetyl-CoA is diverted to increased cholesterol biosynthesis, resulting in hypercholesterolemia. Diabetes mellitus is characterized in large part by glycosuria, hypercholesterolemia, and often ketoacidosis. The accelerated atherosclerotic process seen in diabetics is associated with hypercholesterolemia (Boyle et al., supra.).

Chromium functions as a cofactor for insulin. It binds to the insulin receptor and potentiates many, and perhaps all, of its functions (Boyle et al., supra.). These functions include, but are not limited to, the regulation of carbohydrate and lipid metabolism. (Present Knowledge in Nutrition, supra, at p. 573-577). The introduction of inorganic chromium compounds per se into individuals is not particularly beneficial. Chromium must be converted endogenously into an organic complex or must be consumed as a biologically active molecule. Only about 0.5% of ingested inorganic chromium is assimilated into the body (Recommended Daily Allowances, Ninth Revised Edition, The National Academy of Sciences, page 160, 1980). Only 1-2% of most organic chromium compounds are assimilated into the body.

U.S. Pat. No. Re. 33,988 discloses that when selected essential metals, including chromium, are administered to mammals as exogenously synthesized coordination complexes of picolinic acid, they are directly available for absorption without competition from other metals. This patent describes a composition and method for selectively supplementing the essential metals in the human diet and for facilitating absorption of these metals by intestinal cells. These complexes are safe, inexpensive, biocompatible, and easy to produce. These exogenously synthesized essential metal coordination complexes of picolinic acid (pyridine-2-carboxylic acid) have the following structural formula:

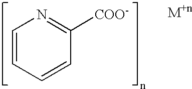

wherein M represents the metallic cation and n is equal to the cation's valence. For example, when M is Cr and n=3, then the compound is chromic tripicolinate. Other chromium picolinates disclosed include chromic monopicolinate and chromic dipicolinate.

The U.S. Recommended Daily Intake (RDI) of chromium is 120 μg. U.S. Pat. No. 5,087,623, the entire contents of which are hereby incorporated by reference, describes the administration of chromic tripicolinate for the treatment of adult-onset diabetes in doses ranging from 50 to 500 μg. International Patent Application No. WO96/35421 discloses the use of high doses of chromic tripicolinate (providing 1,000-10,000 μg chromium/day) for reducing hyperglycemia and stabilizing the level of serum glucose in humans with Type 2 diabetes. U.S. Pat. Nos. 5,789,401 and 5,929,066, the entire contents of which are hereby incorporated by reference, disclose a chromic tripicolinate-biotin composition and its use in lowering blood glucose levels in humans with Type 2 diabetes.

U.S. Pat. Nos. 5,087,623; 5,087,624; and 5,175,156, the entire contents of which are hereby incorporated by reference, disclose the use of chromium tripicolinate for supplementing dietary chromium, reducing hyperglycemia and stabilizing serum glucose, increasing lean body mass and reducing body fat, and controlling blood serum lipid levels, including the lowering of undesirably high blood serum LDL-cholesterol levels and the raising of blood serum High Density Lipid (HDL)-cholesterol levels, the so-called “good” cholesterol. U.S. Pat. Nos. 4,954,492 and 5,194,615, the entire contents of which are hereby incorporated by reference, describe a related complex, chromic nicotinate, which is also used for supplementing dietary chromium and lowering serum lipid levels. Picolinic acid and nicotinic acid are position isomers having the following structures:

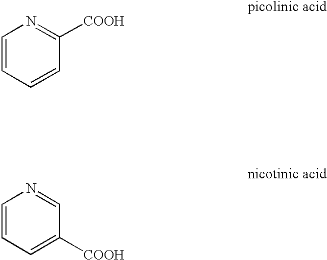

Nicotinic acid and picolinic acid form coordination complexes with monovalent, divalent and trivalent metal ions and facilitate the absorption of these metals by transporting them across intestinal cells and into the bloodstream. Chromium absorption in rats following oral administration of CrCl₃ was facilitated by the non-steroidal anti-inflammatory drugs (NSAIDs) aspirin and indomethacin (Davis et al., J Nutrition Res. 15:202-210, 1995; Kamath et al., J. Nutrition 127:478-482, 1997). These drugs inhibit the enzyme cyclooxygenase which converts arachidonic acid to various prostaglandins, resulting in inhibition of intestinal mucus formation and lowering of intestinal pH which facilitates chromium absorption.

U.S. Pat. No. 4,315,927 discloses that when selected essential metals are administered to mammals as exogenously synthesized coordination complexes of picolinic acid, they are directly available for absorption without competition from other metals. These complexes are safe, inexpensive, biocompatible and easy to produce.

Biotin is the prosthetic group for a number of carboxylation reactions, the most notable being pyruvate carboxylase which is involved in gluconeogenesis and replenishment of the citric acid cycle, and acetyl CoA carboxylase which plays a role in fatty acid biosynthesis. The safe and adequate recommended daily intake of biotin is 100-300 μg, although no side effects or toxicities were noted in previous clinical studies with oral biotin intakes of up to 200 mg daily (Mock et al, in Present Knowledge in Nutrition, seventh edition, Ziegler, E. et al., eds., ILSI Press, Washington, D.C., 1996, pp. 220-235). Supranutritional doses of biotin have been shown to have therapeutic utility in the treatment of various disease states such as diabetes. High-dose oral or parenteral biotin, for example, has been shown to improve oral glucose tolerance in diabetic KK mice (Reddi et al., Life Sci., 42:1323-1330, 1988), rats made diabetic by injection with streptozotocin (Zhang et al., 16th International Congress of Nutrition, Montreal, 1997, abstract book, p. 264) and in pre-diabetic Otsuka Long-Evans Tokushima Fatty rats (Zhang et al., J. Nutr. Sci. Vitaminol. 42:517-526, 1996).

In a clinical study, Coggeshall et al. (Ann. N.Y. Acad. Sci., 447:387-392, 1985) demonstrated that a daily oral dose of biotin of 16 mg lowered fasting plasma glucose levels in Type I diabetics in whom insulin injections had been temporarily discontinued. Maebashi et al. (J. Clin. Biochem. Nutr. 14:211-218, 1993) showed that administration of 3 mg biotin three times per day to poorly-controlled type 2 diabetics resulted in improved pancreatic beta cell function as evidenced by the fact that fasting insulin levels did not decline in biotin-treated subjects despite the sharp decline in glucose levels.

There is a constant need for effective treatments for lowering LDL and triglyceride levels while increasing HDL levels in a subject. The present invention addresses this need by providing a safe, inexpensive, drug-free therapeutic agent. A method of reducing and reversing dyslipidemia without patient side effects would present a substantial advancement in prevention and treatment of a variety of disease states.

### SUMMARY OF THE INVENTION

The present invention is directed to improved insulin sensitivity and blood cholesterol levels in an individual. Accordingly, in one aspect of the invention, a method for treating dyslipidemia including administering to an individual in need thereof between about 25 and 2,000 micrograms per day of a chromium complex in combination with between about 25 μg and 20 mg per day of biotin is provided. Advantageously, the amount of chromium complex administered per day is between about 300 and 1,000 micrograms per day. In preferred embodiments, between about 150 μg and 5 mg biotin are administered per day in order to lower blood cholesterol.

The chromium complex of the present invention may include chromium picolinate, chromic tripicolinate, chromium nicotinate, chromic polynicotinate, chromium chloride, chromium histidinate, or chromium yeasts.

Preferably, the chromium complex is in a pharmaceutically acceptable carrier. Similarly, it is preferred that the biotin likewise is in a pharmaceutically acceptable carrier.

Optionally, the chromium complex and biotin are orally administered. However, in some aspects of the invention, the chromium complex and biotin are parenterally administered.

In yet another aspect of the invention, certain chelating agents may be added to facilitate absorption of the chromium complex. In one aspect of the invention, picolinic acid is administered to an individual. In another aspect, nicotinic acid is administered to an individual. In still another aspect, both picolinic and nicotinic acid are administered to an individual in order to treat dyslipidemia.

In one aspect of the invention, a method of treating hypercholesterolemia in an individual is disclosed. The method may include identifying an individual presenting with hypercholesterolemia; and administering to the individual an effective dose of a chromium complex and biotin. The effective dose of chromium complex may be between about 25 and 2,000 micrograms per day and preferably, the effective dose of chromium complex is between about 300 and 1,000 micrograms per day. The effective dose of biotin may be between about 25 μg and 20 mg per day of biotin. Advantageously, the effective dose of biotin s between about 150 μg and 5 mg biotin per day.

In some aspects of the invention, the method of treating hypercholesterolemia may additionally include the administration of either picolinic acid, nicotinic acid, or both picolinic and nicotinic acid.

In yet another aspect of the invention, a method of increasing levels of HDL cholesterol in the blood including administering to an individual in need thereof between about 25 and 2,000 micrograms per day of a chromium complex in combination with between about 25 μg and 20 mg per day of biotin is provided. Advantageously, the amount of chromium complex administered is between about 300 and 1,000 micrograms per day. Preferably, the amount of biotin administered is between about 150 μg and 5 mg per day of biotin.

The chromium complex utilized for increasing levels of HDL cholesterol in the blood may include chromium picolinate, chromic tripicolinate, chromium nicotinate, chromic polynicotinate, chromium chloride, chromium histidinate, or chromium yeasts.

Advantageously, the chromium complex is in a pharmaceutically acceptable carrier. Similarly, in preferred aspects of the present invention, the biotin is likewise in a pharmaceutically acceptable carrier.

The chromium complex may be orally or parenterally administered. Likewise, the biotin may be orally or parenterally administered.

In one aspect of the invention, the method of increasing HDL cholesterol levels in the blood includes administering chelating agents such as picolinic acid or nicotinic acid. In some aspects of the invention, both picolinic acid and nicotinic acid are added to increase HDL cholesterol levels in the blood.

In still another aspect of the invention, a composition consisting essentially of an effective dose of chromium complex and biotin is provided, wherein the ratio of chromium complex to biotin is from about 1:1,000 to about 100:1 (w/w). Advantageously, the chromium complex may include chromium picolinate, chromic tripicolinate, chromium nicotinate, chromic polynicotinate, chromium chloride, chromium histidinate, or chromium yeasts.

In still another aspect of the invention, a method of reducing post prandial hyperglycemia in an individual is provided. Advantageously, the method includes administering to an individual in need thereof between about 25 and 2,000 micrograms per day of a chromium complex in combination with between about 25 μg and 20 mg per day of biotin is provided. Advantageously, the amount of chromium complex administered per day is between about 300 and 1,000 micrograms per day. In preferred embodiments, between about 150 μg and 5 mg biotin are administered per day in order to reduce post-prandial hyperglycemia.

In yet another aspect of the invention, a method of lowering the glycemic index of a food is provided. The method includes supplementing the food with an effective amount of a chromium complex in combination with an effective amount of biotin.

### BRIEF DESCRIPTION OF THE DRAWINGS

FIG. 1 illustrates the insulin signaling cascade.

FIG. 2 is a graph demonstrating 2-deoxyglucose uptake with chromium picolinate and biotin.

FIG. 3 is a graphic representation of glycogen synthesis in human skeletal muscle culture when incubated with chromium picolinate and biotin.

FIG. 4A is a bar graph depicting glycogen synthesis.

FIG. 4B is a bar graph illustrating gene expression (mRNA).

FIG. 5 is a chart depicting the study design for evaluation of the effect of chromium and biotin on insulin sensitivity in rats.

FIG. 6 is a bar graph representing the results of co-administration of chromium and biotin on glucose disposal in a rat model.

FIG. 7 is a bar graph illustrating the effects of chromium picolinate on fasting plasma insulin levels in obese rats at baseline and end of study.

FIG. 8A is a graphic depiction of the results of the intraperitoneal glucose tolerance test in obese rats and the effects of chromium picolinate on glucose tolerance.

FIG. 8B demonstrates the insulin response observed after treatment with chromium picolinate.

FIG. 9 is a bar graph demonstrating the effects on insulin sensitivity in obese rats as compared to lean rats with the administration of chromium picolinate versus the control.

FIG. 10 is a line graph representing the effects observed over time on the cholesterol levels of rat models treated with a variety of chromium and biotin protocols.

FIG. 11 is a bar graph depicting the change of cholesterol over time in various treatment protocols involving the administration of high and low doses of chromium and biotin, alone or in combination.

FIG. 12A is a bar graph demonstrating the HDL-cholesterol profile in JCR rats treated with chromium picolinate.

FIG. 12B is a bar graph detailing the cholesterol/HDL ratio in JCR rats treated with chromium picolinate.

FIG. 13 is a bar graph illustrating the HDL/cholesterol ratio in treated and untreated JCR rats.

FIG. 14 is a bar graph representing the change in HDL levels over time of JCR rats that have been administered various combinations of chromium and biotin.

FIG. 15 is a line graph charting the HDL profile of test animals administered various doses of chromium and biotin, either alone or in combination.

FIG. 16 is a line graph detailing the change in triglyceride levels over time in rats treated with chromium and biotin, either alone or in combination.

FIG. 17 is a bar graph illustrating the change in triglyceride profile in rats administered various doses of chromium, biotin, or both.

FIG. 18 is a bar graph illustrating the effect of chromium picolinate and biotin added beverage on glycosylated hemoglobin levels.

FIG. 19 is a bar graph demonstrating the effect of chromium picolinate and biotin added beverage on blood glucose.

FIG. 20 is a graphic representation of the effect of chromium picolinate and biotin on fatigue.

### DETAILED DESCRIPTION OF THE PREFERRED EMBODIMENT

The disclosed invention relates to methods and compositions for the treatment of hypercholesterolemia and post prandial hyperglycemia. Additionally, the compositions and methods of the present invention are useful in enhancing insulin sensitivity, reducing hyperglycemia in an individual, and lowering the glycemic index of food.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the invention herein described. As used herein, the term “chromium complexes” or “chromium complex” includes, without limitation, chromium picolinate, chromic tripicolinate, chromium nicotinate, chromic polynicotinate, chromium chloride, chromium histidinate, and chromium yeasts.

A primary basis of the present invention is the novel and unexpected discovery that the co-administration of an effective dose of a chromium complex in combination with biotin produces a synergistic improvement in insulin sensitivity. The co-administration of chromium and biotin can facilitate the treatment and recovery of individuals suffering from a variety of medical conditions. The conditions contemplated as treatable under the present invention result from a disparate number of etiological causes. Nevertheless, they share a common feature in that their pathological conditions are either caused or exacerbated by insulin insensitivity or post prandial hyperglycemia. Moreover, the administration of a chromium complex in combination with an effective dose of biotin provides an effective reduction of hypercholesterolemia, increase in HDL cholesterol in the blood, improvement of insulin sensitivity, and reduction of hyperglycemia such as post prandial hyperglycemia. This reduction is markedly greater than what would be expected when either component is administered alone, thus indicating a synergistic effect. Additionally, the co-administration of a chromium complex and biotin has been observed to lower the glycemic index of foods.

Insulin resistance is a key pathogenic parameter of Type 2 diabetes, and clinical interventions that improve insulin sensitivity are considered cornerstones in the management of the disease. In addition, the relationship of insulin resistance to cardiovascular disease and its associated risk factors has been well established over the past few years. Therefore, with the recent release of numerous medications, current treatment of Type 2 diabetes is aimed toward achieving “clinical insulin sensitization.” This concept is based on the established clinical goal of lowering blood glucose in an effort to reduce microvascular complications (i.e. eye, kidney, and nerve disease) with minimal endogenous insulin stimulation or the lowest exogenous insulin dosing possible. Because of such a clinical concept, combination therapy for treatment of Type 2 diabetes is now considered the standard of care in clinical management. Combinations of pharmacologic agents (such as sulfonylureas/metformin, sulfonylureas/glitazones, and metformin/glitazones) are highly effective pharmacologic interventions that appear to lower both glucose and insulin levels. Further, there is evidence that triple drug therapy (e.g. sulfonylureas/metformin/glitazones) can lower clinical glycemia in addition to lowering insulin levels. Because of the success of these clinical formulations, pharmacologic agents that have a clinical effect to both lower glucose and improve insulin action will be considered very favorably in the future management of Type 2 diabetes.

In contrast to pharmacologic therapy, nutritional intervention is a very attractive approach to treatment of insulin resistance and therefore, glucose control of Type 2 diabetics. In this regard, there is strong evidence to suggest that supplemental chromium complexes such as chromium picolinate (CrPic) may favorably improve insulin sensitivity and glycemic control in human trials. Specifically, this has been demonstrated in a study of Chinese diabetics, where CrPic significantly lowered glucose and insulin levels. Anderson et al. Elevated intakes of supplemental chromium improve glucose and insulin variables in individuals with type 2 diabetes. Diabetes. 46: 1786-1791 (1997). Recently, it has been demonstrated that CrPic at 1000 μg per day can enhance insulin sensitivity in a cohort of subjects representing the obese, pre-diabetic human population. Cefalu, W. T. et al. Effects of chromium picolinate on insulin sensitivity in vivo. J. Trace Elem. Exp. Med. 12: 71-83 (1999). Similarly, CrPic has a very robust effect on improving insulin sensitivity and lipid profiles in an obese, hyperinsulinemic, insulin resistant rodent model. Wang et al. Chromium picolinate enhances insulin sensitivity in an animal model for the metabolic syndrome: the obese, insulin resistant JCR:LA-corpulent rat. Diabetes; In press (abstract to be presented at the Annual Meeting of the ADA, June 2000) (2000). This strongly suggests that subjects with the components of “Syndrome X” (i.e. central obesity, dyslipidemia, and insulin resistance) may favorably respond to chromium supplementation.

In addition to monotherapy treatments with a chromium complex such as chromium picolinate, recent research strongly suggests that other nutrients, such as biotin, can enhance the effect of chromium observed on glucose uptake. Specifically, the combination of a chromium complex with biotin greatly enhances glucose uptake in a human skeletal muscle culture. Without being limited to a particularly theory, it is believed that the cellular mechanism by which a chromium complex may enhance insulin sensitivity is improved by enhancing glycogen synthesis. The importance of this observation in understanding the mechanism by which chromium enhances insulin sensitivity is heightened by the fact that the aspect of insulin resistance that has been the most described is the inefficient glucose uptake and utilization in response to insulin sensitivity in insulin sensitive tissues. This is represented by a reduction in the insulin-stimulated storage of glucose as glycogen in both muscle and liver.

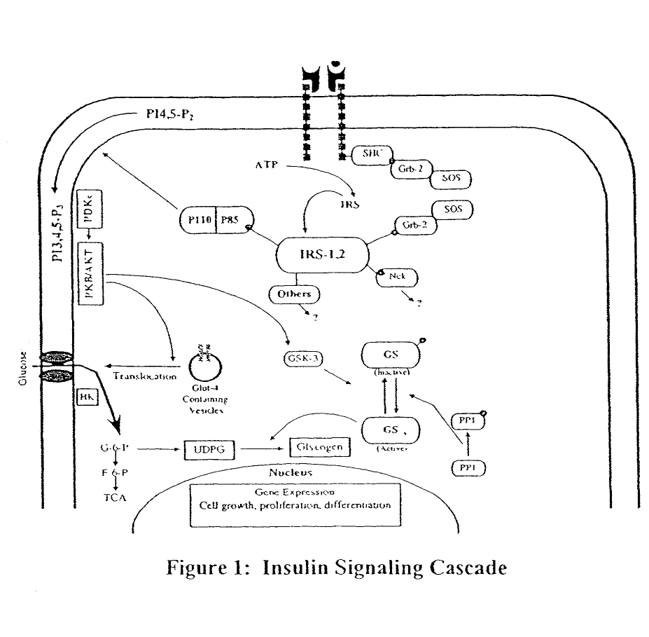

The specific cellular defects that characterize insulin resistance have not been fully characterized. However, FIG. 1 outlines currently accepted pathways in the insulin signaling cascade and specifically outlines pathways involved in glucose transporter (Glut-4) translocation and glucogen synthesis. These two cellular parameters are altered in insulin resistant states and overcoming these defects leads to an increase in insulin sensitivity. In this regard, the present invention is directed to exploring the role of biotin and chromium complexes in enhancing cellular signaling, leading to enhanced target insulin both in vitro and in vivo.

Post prandial hyperglycemia is characterized by high glucose levels after meals, which do not return to normal levels after a period of time (e.g. two to three hours after meals). Acute glucose elevations after meal ingestion are associated with a variety of glucose-mediated tissue defects such as oxidative stress, glycation, and advanced glycation end product formation, which have far-reaching structural and functional consequences for virtually every human organ system. Lowering glycosylated hemoglobin to levels that prevent or delay these complications can be achieved only by reducing both post prandial and fasting plasma glucose levels. Accordingly, in some embodiments, the present invention includes the co-administration of a chromium complex in concert with an effective amount of biotin to delay the digestion and absorption of carbohydrates, thereby diminishing the post prandial surge in blood glucose levels without loss of calories.

In addition to reducing the post prandial surge in blood glucose levels, we have observed that the co-administration of a chromium complex and biotin acts to lower the glycemic index of a food. As detailed above, when consumed, foods with higher glycemic indexes such as foods containing carbohydrates can cause a surge in blood sugar followed by a drop over time. One of the surprising discoveries of the present invention is the observation that the administration of a chromium complex in concert with a biotin to a food reduces the glycemic index of the food. The co-administration of a chromium complex and biotin prevent the sharp elevation in glucose response shortly after a food is ingested. Additionally, the “highs” and subsequent “lows” attendant to surges and drops in blood sugar levels are reduced.

Accordingly, in one embodiment, a method of reducing the glycemic index of food is provided. As used herein, the term “food” includes any material consisting essentially of protein, carbohydrate, and fat used in a body to sustain growth, repair, and vital processes and to furnish energy. In particular, food refers to all solid, semi-solid, and liquid nourishment found in the following recognized food groups: the bread, cereal, rice, and pasta group; the vegetable group; the fruit group; the milk, yogurt, and cheese group; the meat, poultry, fish, dry beans, eggs, and nuts group (hereinafter referred to as the meat group); the fats and oils group, and the processed foods group which contains such items as sugars, candies, cakes, salty processed snack foods, sugar sweetened beverages such as soft drinks, and the like.

The reduction of the glycemic index of food is accomplished by administering a chromium complex and biotin to a food. The administration can be accomplished in a variety of ways. For example, the chromium complex and biotin can be incorporated into a food product as the food is being prepared. In other words, the chromium complex and biotin are added at the same time the other component ingredients of a food item as are combined. Alternatively, the chromium complex and biotin may be added after a food product has been prepared. The chromium complex and biotin may be formulated as a powder or liquid (specific formulations are detailed below) and distributed over the surface of an already prepared food. The chromium complex and biotin can be added alone or combined with other ingredients prior to administering the chromium and biotin to a food item. For example, the chromium complex and biotin may be mixed with a sweetening agent and sprinkled over a food such as cereal. Other methods of administration may also be suitable.

The compounds of the present invention can be administered separately or as a single composition (i.e., combined). If administered separately, the compounds should be given in a temporally proximate manner such that the desired glycemic index lowering effect is enhanced. More particularly, the compounds may be given within one hour of each other. In one embodiment, the chromium complex and biotin are added substantially simultaneously to the food item.

In addition, the present invention contemplates compositions and methods that are efficacious in ameliorating a variety of conditions wherein insulin sensitivity, or lack thereof, plays an active, detrimental role in the development of the disease. Such conditions include but are not limited to: diabetes, Syndrome X, insulin resistance and related detrimental effects, hyperinsulinemia, hyperglyceridemia, depression, premenstrual syndrome (PMS), premenstrual dysphoric disorder (PMDD), obesity, cardiovascular disease, osteoporosis, periodontal disease and polycystic ovary syndrome (PCOS), as well as other conditions wherein insulin sensitivity can play an important role. In particularly preferred embodiments, a method of lowering the amount of LDL cholesterol in the blood is provided. In most preferred embodiments, methods of increasing the level of HDL is provided. Advantageously, the co-administration of chromium and biotin in an effective dose is useful in the treatment and prevention of hypercholesterolemia.

In some embodiments, the compositions and methods disclosed herein are useful in improving body composition, decreasing body fat, increasing lean muscle mass, enhancing muscle growth and repair, and improving athletic performance and endurance. It is proposed that the co-administration of an effective dose of a chromium complex in concert with an effective dose of biotin promotes insulin sensitivity, thereby enhancing body composition in an individual.

In some embodiments of the present invention, the compositions and methods have utility for promoting animal health. In addition to the above-referenced health benefits, the co-administration of a chromium complex and biotin can prevent hoof disease and lower the amount of fat and cholesterol in meat, milk, eggs, and other animal products, for example, birds and mammals may advantageously be treated. Additionally, the formulations of the present invention are useful in increasing milk production, egg laying, and litter size.

In a preferred embodiment, the present invention contemplates using chromium complexes in combination with biotin to achieve a beneficial reduction in LDL and increase in HDL cholesterol levels in the blood. The compounds of the present invention can be administered to an individual separately or as a single composition. Advantageously, an individual is administered a pharmaceutically effective dose of a chromium complex such as chromium picolinate. In one embodiment, the biotin is administered substantially simultaneously. In an alternative embodiment, the chromium complex is administered first and then the biotin is added second. In yet another embodiment, the biotin is administered first. If administered separately, the compounds should be given in a temporally proximate manner, e.g. within a twenty-four hour period, such that the reduction of hypercholesterolemia is enhanced. More particularly, the compounds may be given within one hour of each other. The administration can be by any of the methods of administration described below or by drug delivery methods known by one of skill in the art.

The synthesis of chromic picolinates is described in U.S. Pat. No. 5,087,623, the entire contents of which are hereby incorporated by reference. Biotin and chromium complexes such as chromium tripicolinate are commercially available from health food stores, drug stores and other commercial sources. In order to lower LDL cholesterol levels and increase HDL levels, it is anticipated that the dosage range of chromium administered to an individual will be at least about 25 μg/day. Preferably, the amount of chromium will be between about 25 and 2,000 μg/day. More preferably, the amount of chromium is between about 300 and 1,000 μg/day. Most preferably, the amount of chromium is between about 400 and 1,000 μg/day. In a particularly preferred embodiment, the amount of chromium is between about 600 and 1,000 μg/day. With regard to the biotin component of the combination therapy, the daily dosage is at least 25 μg. Preferably, the amount of biotin is between about 25 μg and 20 mg per day. More preferably, the daily dosage of biotin is between about 150 μg to 10 mg. Most preferably, the daily dose of biotin is between about 300 μg and 5 mg. Note that these doses are based on a 70 kg adult human, and that the dose can be applied on a per-kilogram basis to humans or animals of different weights.

The preferred daily dose of a chromium complex for the reduction of the glycemic index of food will be at least about 25 μg of a chromium complex to be administered to a food item. In a preferred embodiment, the amount of chromium is between about 50 μg and 1,000 μg of chromium. In particularly preferred embodiments, the amount of chromium is about 75 μg, 100 μg, 150 μg, 200 μg, 250 μg, 300 μg, 350 μg, 400 μg, 450 μg, 500 μg, 550 μg, 600 μg, 650 μg, 700 μg. With reguard to the daily dose of the biotin component, the preferred amount of biotin to be administered to a food item is at least 25 μg, preferably between 50 μg and about 10 g, and more preferably between about 100 μg and 3 grams. The amount of chromium complex and biotin added to a particular food item will depend on the glycemic index and energy-density of the food item, the serving size of the food item, and the number of servings of that particular food item expected to be consumed in one day. In general, food items having a higher glycemic index and/or higher energy density, and/or food items which are expected to be consumed in larger serving sizes and/or several times per day, will require the addition of higher amounts of chromium complex and biotin to achieve the desired effect.

While the chromium complexes aid in the absorption of chromium by intestinal cells, in some embodiments, uncomplexed chelating agents are advantageously included in the compositions to facilitate absorption of other ingested chromium as well as other metals including, but not limited to, copper, iron, magnesium, manganese, and zinc. Suitable chelating agents include picolinic acid, nicotinic acid, or both picolinic acid and nicotinic acid. Thus, the compositions of the disclosed invention are readily absorbable forms of chromium which also facilitate absorption of other essential metals in the human diet.

The chromium complexes of the disclosed invention have the same uses as described for chromic tripicolinate in U.S. Pat. Nos. 5,087,623, 5,087,624 and 5,174,156, namely supplementing dietary chromium, lowering blood glucose levels in diabetics, lowering serum lipid levels and increasing lean body mass. Additionally, the chromium picolinate of the present invention act to treat symptoms associated with diabetes.

Methods of lowering the glycemic index of food is likewise provided.

Advantageously, the chromium complexes are synthetic. The synthesis and use of chromium picolinates, for example, is described in U.S. Pat. No. Re 33,988 and U.S Pat. No. 5,087,623. Chromic tripicolinate is available from health food stores, drug stores and other commercial sources. The synthesis and use of chromic polynicotinate is described in U.S. Pat. No. 5,194,615.

The chelating agents such as picolinic acid and nicotinic acid are available from many commercial sources, including Sigma-Aldrich (St. Louis, Mo.) (picolinic acid; catalog No. P5503; nicotinic acid; catalog No. PN4126). Preferably, the ratio of the chromium complex to the chelating agent from about 10:1 to about 1:10 (w/w), more preferably from about 5:1 to about 1:5 (w/w). Alternatively, the molar ratio of chromium complex to the uncomplexed chelating agent is preferably 1:1, and may be from about 5:1 to about 1:10.

For oral administration, the chromium complex and biotin may be provided as a tablet, aqueous or oil suspension, dispersible powder or granule, emulsion, hard or soft capsule, syrup, elixir, or beverage. Compositions intended for oral use may be prepared according to any method known in the art for the manufacture of pharmaceutically acceptable compositions and such compositions may contain one or more of the following agents: sweeteners, flavoring agents, coloring agents and preservatives. The sweetening and flavoring agents will increase the palatability of the preparation. Tablets containing chromium complex in admixture with non-toxic pharmaceutically acceptable excipients suitable for tablet manufacture are acceptable. Pharmaceutically acceptable means that the agent should be acceptable in the sense of being compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets may be uncoated or may be coated by known techniques to delay disintegration and absorption in the gastrointestinal tract and thereby provide a sustained action over a longer period of time. For example, a time delay material such as glyceryl monostearate or glyceryl distearate alone or with a wax may be employed.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil. Aqueous suspensions may contain the chromium complex of the invention in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or cetyl alcohol. Sweetening agents, such as those set forth above, and flavoring agents may be added to provide a palatable oral preparation. These compositions may be preserved by an added antioxidant such as ascorbic acid. Dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, may also be present.

Syrups and elixirs may be formulated with sweetening agents, such as glycerol, sorbitol or sucrose. Such formulations may also contain a demulcent, a preservative, a flavoring or a coloring agent.

The chromium complex preparations for parenteral administration may be in the form of a sterile injectable preparation, such as a sterile injectable aqueous or oleaginous suspension. This suspension may be formulated according to methods well known in the art using suitable dispersing or wetting agents and suspending agents. The sterile injectable preparation may also be a sterile injectable solution or suspension in a non-toxic parenterally-acceptable diluent or solvent, such as a solution in 1,3-butanediol. Suitable diluents include, for example, water, Ringer's solution and isotonic sodium chloride solution. In addition, sterile fixed oils may be employed conventionally as a solvent or suspending medium. For this purpose, any bland fixed oil may be employed including synthetic mono or diglycerides. In addition, fatty acids such as oleic acid may likewise be used in the preparation of injectable preparations.

The pharmaceutical compositions may also be in the form of oil-in-water emulsions. The oily phase may be a vegetable oil, such as olive oil or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

The amount of chromium complex/biotin that may be combined with the carrier material to produce a single dosage form will vary depending upon the host treated and the particular mode of administration.

### EXAMPLES

The following examples teach the methods and compositions disclosed herein for improving insulin sensitivity, reducing cholesterol levels, treating post prandial hyperglycemia, and reducing the glycemic index of food through the administration of at least one chromium complex in concert with biotin. These examples are illustrative only and are not intended to limit the scope of the invention disclosed herein. The treatment method described below can be optimized using empirical techniques well known to those of ordinary skill in the art. Moreover, artisans of skill would be able to use the teachings described in the following examples to practice the full scope of the invention disclosed herein.

### Example 1

### Effects of Chromium and Biotin on Insulin Sensitivity

To evaluate the potential cellular mechanism for CrPic's in vivo effect, the role of chromium was evaluated, as monotherapy and in combination with biotin, on glucose uptake in a human skeletal muscle culture (“HSMC”). The value of the HSMC has been reported as representative of an insulin sensitive target tissue and skeletal muscle is the major tissue for glucose disposal in humans. See Henry, R. R. et al. Acquired defects of glycogen synthase activity in cultured human skeletal muscle cells: influence of high glucose and insulin levels. Diabetes, 45: 400-407 (1996).

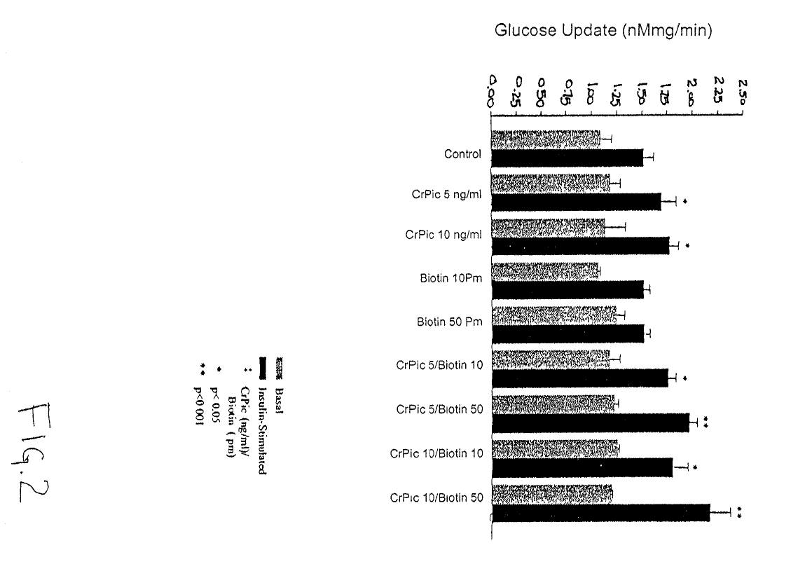

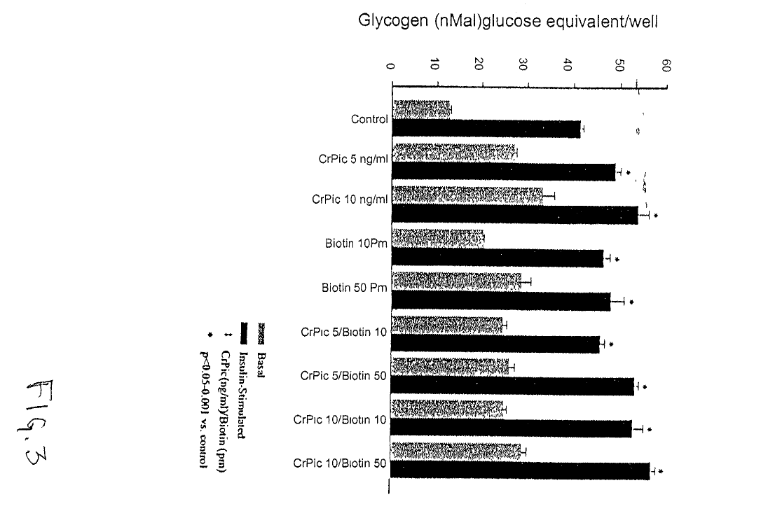

With specific regard to glucose uptake, we have demonstrated that chromium alone enhances glucose uptake in contrast to biotin alone. However, when the HSMC was incubated with both, a synergistic effect on glucose uptake was observed as illustrated in FIG. 2. This observation was further extended to the assessment of glucogen synthesis. The human skeletal muscle cell line was plated and grown in the presence of CrPic, biotin, and combined CrPic/biotin. After incubation, cells were fasted and glycogen synthesis was analyzed at baseline and after insulin stimulation. Chromium and biotin increased glycogen synthesis both at basal and insulin-stimulated conditions as illustrated in FIG. 3. Protein content of IRS-2 was also increased in CrPic +biotin combination.

### Example 2

### Evaluation of the Cellular Mechanism by which CrPic and Biotin Enhance Glycogen Synthesis

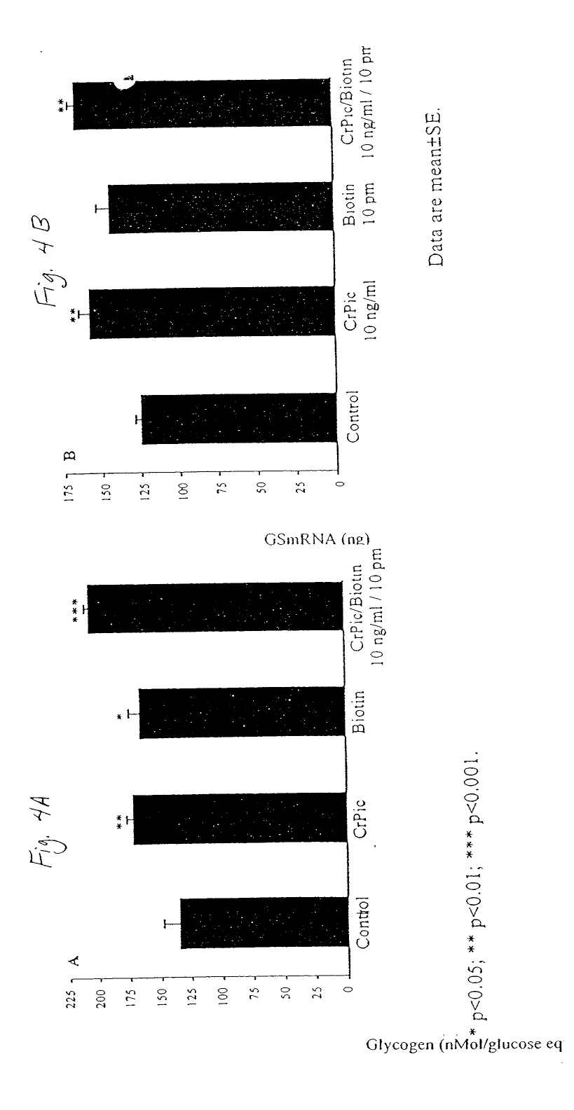

To evaluate the cellular mechanism by which CrPic and biotin enhance glycogen synthesis, glycogen synthetase (“GS”) MRNA and glycogen synthesis were assessed. HSMC was incubated with CrPic at 10 ng/ml, biotin at 10 pm, and both CrPic and biotin. As demonstrated, CrPic and biotin were again observed to enhance insulin stimulated glycogen synthesis as depicted in FIG. 4A. When assessing gene expression, the combination of CrPic and biotin was observed to enhance GS mRNA (see FIG. 4B). When evaluating GS MRNA levels, chromium increased gene expression of GS by 26%, biotin by 15%, and the combination by 33%. This strongly suggests a synergistic effect of chromium and biotin on GS gene expression.

The mechanism proposed is that CrPic and biotin affect the rate of gene transcription of enzymes (particularly GS) involved in mediating the biologic effects of insulin in human skeletal muscle.

### Example 3

### Effect of Chromium and Biotin on Insulin Sensitivity in an Animal Model

In order to test the effects of chromium and biotin, alone and in combination, on insulin sensitivity, a rodent model of “Syndrome X” was employed. Specifically, the JCR rat was utilized as it is a well-established model of obesity and insulin resistance. In addition, this model exhibits clinical components of the insulin resistance syndrome (e.g., dyslipidemia, central obesity).

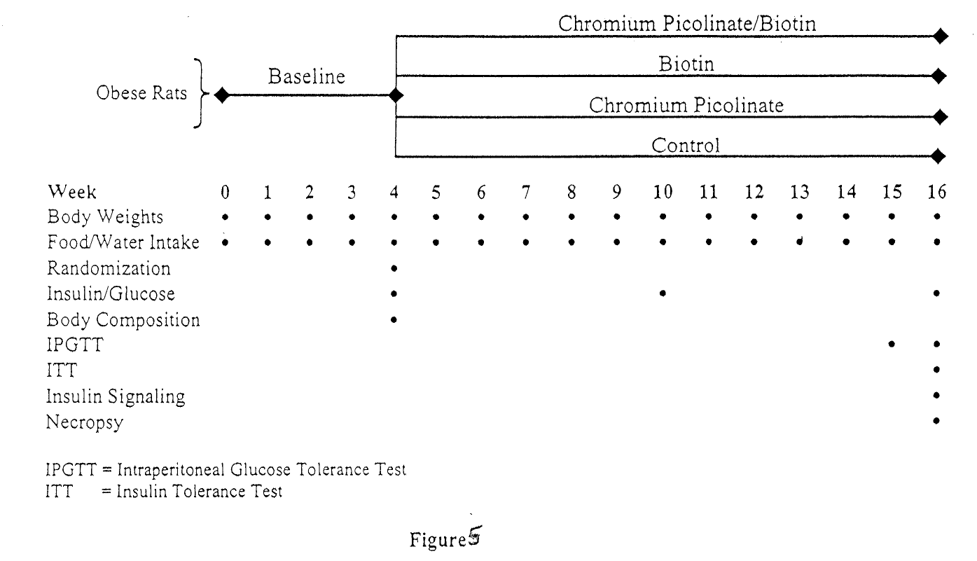

After a baseline phase where accurate weights, body composition, and food intake were assessed, the animals were randomized to either control, CrPic alone, biotin alone, or CrPic/Biotin (See FIG. 5). Animals were weighed weekly and nutrients were given via daily water feeding at a specified dose/kg body weight. A 12-week period of daily treatment of animals was accomplished.

Methods

At specified time points during the study, glucose and insulin assessments were made. Specific assessments of carbohydrate metabolism were determined using an intraperitoneal glucose tolerance test and an insulin tolerance test. Animals were studied at 6 and 12 weeks with randomization. The specific metabolic testing was as follows:

Intraperitoneal Glucose Tolerance Test (IPGTT)

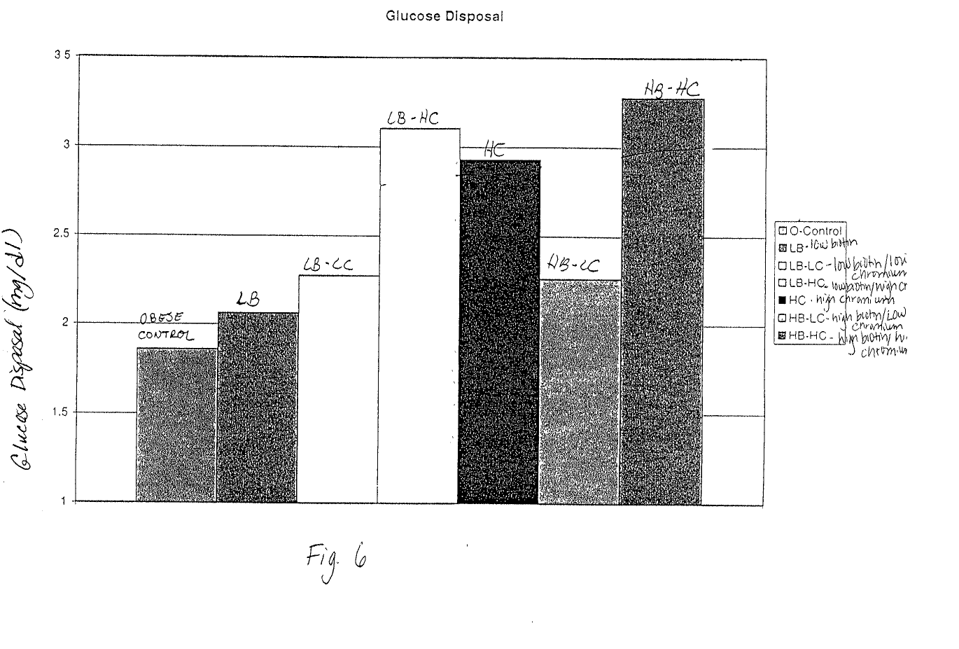

Following baseline glucose and insulin measurements, 1.5 mg D₅₀W per gram of body weight were given intraperitoneally. Tailsticks were initiated at 30, 60, 90, and 120 minutes following administration of glucose. The areas under curve for insulin and glucose were assessed. Eight animals in each group at 12 weeks were studied. The results of the IPGTT are reflected in Table 1. Table 1 details the amount of glucose disposal for animals treated with CrPic and/or biotin at various dosages. Note, “L” represents the lean control; “O-contr” are the obese control rats; “LB” connotes low biotin treatments; “HB” represents high biotin treatments; “LC” and “HC” represent low and high doses of chromium, respectively. FIG. 6 is a graphic representation of the results of the IPGTT studies.

TABLE 1
Glucose Disposal Data for the Chromium
Picolinate + Biotin Animal Study

| Rat # | Cage # | Treated | Body Weight | Glucose Disposal (mg/dl/min) | | |
|---|---|---|---|---|---|---|
| | 1 | L | 375 | 3.61 | O-Control | 1.8585 |
| | 2 | L | 443 | 4.424 | LB | 2.0615 |
| | 3 | L | 420 | 3.114 | LB-LC | 2.273333 |
| | 4 | L | 400 | 3.248 | LB-HC | 3.1005 |
| | 5 | L | 370 | 4.562 | HC | 2.928 |
| | 6 | L | 400 | 4.124 | HB-LC | 2.258667 |
| | 7 | L | 420 | 3.714 | HB-HC | 3.275333 |
| | 8 | L | 400 | 4.177 | | |
| | 9 | O-Contr | 751 | 2.005 | | |
| | 10 | O-Contr | 859 | 1.876 | | |
| | 11 | O-Contr | 900 | 1.632 | | |
| | 12 | O-Contr | 810 | 1.921 | | |
| | 13 | 9 LB | 866 | 2.21 | | |
| | 14 | 9 LB | 812 | 2.392 | | |
| | 15 | 10 LB | 867 | 1.876 | | |
| | 16 | 10 LB | 816 | 1.768 | | |
| | 17 | 11 LB LC | 816 | 2.55 | | |
| | 18 | 11 LB LC | 720 | 2.192 | | |
| | 19 | 12 LB LC | 953 | 2.078 | | |
| | 20 | 13 LB HC | 955 | 3.12 | | |
| | 21 | 13 LB HC | 795 | 2.879 | | |
| | 22 | 14 LB HC | 825 | 3.24 | | |
| | 23 | 14 LB HC | 837 | 3.163 | | |
| | 24 | 15 HC | 830 | 2.928 | | |
| | 25 | 17 HB | 758 | 1.92 | | |
| | 26 | 18 HB | 935 | 2.329 | | |
| | 27 | 18 HB | 856 | 1.879 | | |
| | 28 | 19 HB LC | 815 | 2.34 | | |
| | 29 | 20 HB LC | 872 | 2.412 | | |
| | 30 | 20 HB LC | 840 | 2.024 | | |
| | 31 | 21 HB HC | 960 | 3.211 | | |
| | 32 | 22 HB HC | 820 | 3.311 | | |
| | 33 | 22 HB HC | 780 | 3.304 | | |
| | 1 | 23 Cont | 750 | | | |
| | 2 | 24 LC | 720 | 2.334 | | |
| | 3 | 24 LC | 750 | 2.109 | | |
| | 4 | 26 LB | 835 | 1.875 | | |
| | 5 | 27 LB LC | 748 | 2.31 | | |
| | 6 | HB LC | 722 | 2.2 | | |
| | 7 | 29 HC | 855 | 3.25 | | |
| | 8 | 29 HC | 795 | 3.224 | | |
| | 9 | 30 HC | 705 | 3.011 | | |
| | 10 | 30 HC | 786 | 2.821 | | |
| | 11 | 31 HB | 700 | 1.772 | | |
| | 12 | 31 HB | 788 | 1.993 | | |
| | 13 | 34 HB HC | 727 | 3.04 | | |
| | 14 | LC | | 2.066 | | |

Insulin Tolerance Test (ITT)

Following baseline glucose assessment, insulin (5 units per kg body weight) was given intravenously. Repeat glucose assessments occurred at 5, 15, and 30 minute intervals following insulin administration. The rate of glucose disappearance was measured. Again, eight animals in each group at 12 weeks were studied.

Body Composition

Total body fat was determined at baseline and end of study. For this purpose, the animal was anesthetized with Metaphane, an inhaled gas that induces anesthesia in less than 30 seconds and has been found to be very well tolerated by rodents. The system to measure body composition is the TOBEC (total body electrical conductivity measuring device). The essential principle in this method is that an electromagnetic field is distorted as a direct function of the content of mineral containing tissues (i.e. lean tissue). The machine does not use radiation and poses no biological hazard. Once anesthetized, the rat was placed inside the TOBEC machine with no restraint, readings were taken, and measurements were performed.

Skeletal Muscle Biopsy

At the end of the study, biopsies were made of the vastus lateralis muscle after insulin stimulation. The determination of glycogen content and gene expression studies were performed as follows:

1. Glycogen Content of Skeletal Muscle: Glycogen hydrolysis and glucose determination in the skeletal muscle biopsy were performed according to Gomez-Lechon with some modifications. Gomez-Lechon, M. J. et al. A microassay for measuring glycogen in 96-well-cultured cells. Anal. Biochem. 102: 344-352 (1980). After biopsy, the skeletal muscle cells were washed three times with PBS (pH 7.4). 100 nM insulin and 30 mM glucose were added to media (only SKBM), or added to the same volume of 0.9% NaCl as basal incubation for 2 hr. After extensive washes in ice-cold PBS, all liquid was removed and thereafter, 200 μl of 0.2M sodium acetate buffer, pH 4.8 was added to each well, sonicated using a high-intensity ultrasonic processor (VIR Sonic 60) at setting 7 for 20 sec. 50 μl/well aliquots of homogenates were taken for DNA assay, as described in Labarca, C. and Paigen, K. A simple, rapid, and sensitive DNA assay procedure. Anal. Biochem 102: 344-352 (1980). Amyloglucosidase was added to each well at final concentration at 500 mU/well. Plates were incubated at 40° C. for 2 hr with shaking to prevent the sediment of glycogen-protein aggregates. The products of enzyme digestion were collected to 1.5 ml microcentrifuge tubes and centrifuged at 3000 rpm for 10 min. For the glucose assay, 50 μl/well aliquots of the above supernatants were transferred to a 96 well plate and 150 μl of assay solution (20 u/liter peroxidase, 10 u/liter glucose oxidase, and 1 g/liter ABST were added. Samples were incubated at room temperature in the dark for 30-40 minutes. The intensity of the color reaction was measured at 405 nm using a microplate reader. A blank of the reaction was performed by incubation of cell homogenates without glucoamylase; this value represented the free glucose content and was subtracted from the total glucose obtained after enzymatic hydrolysis. A standard curve was constructed with known amounts of rabbit liver glycogen and processed as test samples. The glycogen contents were expressed as nM glucose equivalent/well after corrected by DNA concentration.

2. Detection of Hexokinase and Glycogen Synthase Kinase-3 (“GSK-3”) proteins: Western blot analysis was performed by the method of Burnett. Burnett, W.N. Western Blotting: electrophoretic transfer of proteins from sodium dodecyl sulfate—polyacrylamide gels to unmodified nitrocellulose and radiographic detection with antibody and radioiodinated protein A. Anal. Biochem. 112: 195-203 (1981). After washing the skeletal muscle cells with PBS three times, the cells were centrifuged at 600×g for 5 min. 50 μl of buffer B (100 mM Tris/HCl pH 7.4 containing 100 mM sodium pyrophosphate, 100 mM sodium fluoride, 5 mM EDTA, 2 mM sodium orthovanadate, 1 mM PMSF, 20 ug/ml aprotinin and 1% triton x-100) were added to microcentrifuge tubes and sonicated for 20 sec as described above. Sonicates were kept on ice for 30 min. and centrifuged 15000×g for 10 min. Protein concentration of supernatant were measured. Aliquots of supernatants that normalize to amount of protein were diluted 1:1 in 2×Lamelli's buffer and heated for 5 min at 95° C. Proteins were separated on 8% SDS-PAGE gels and then transferred to nitrocellulose. HK protein was measured by incubating nitrocellulose sheets with anti-HK polyclonal antibody (raised from sheep at 1:250 dilution in TBS buffer containing 3% BSA) overnight at 4° C., then 15 min×4 TBS washes, followed by incubation with anti-sheep antibody conjugated with horseradish peroxidase (HRP, 1:75000 dilution). GSK-3 proteins were identified using a monoclonal IgG (0.5 ug/ml) raised against a synthetic peptide (CKQLLHGEPNVSYICSRY), which recognizes GSK-3 at 46-51 kDa (Upstate, Lake Placid, N.Y.). The second antibody was anti-mouse IgG conjugated with HRP 1:5000 dilution (Sigma, St. Louis, Mo.). After extensive washing with TBS, immune complexes were detected using an enhanced chemiluminescence kit. The bands on the films were quantified by Alpha Imager 2000 (San Leandro, Calif.).

RNA Preparation: Total RNA from the skeletal muscle biopsy was isolated using guanium thicyanate, phenol-chloroform extraction, and alcohol precipitation. RNA sample was quantified by spectrophotometer. The absorption ratio (260:280 nm) was between 1.8 to 2.0 for all preparations and the integrity of the RNA was verified on agarose gel colored with ethidium bromide. For RT-PCR, fresh isolated total RNA was used.

GS mRNA Level Analysis: GS mRNA levels were analyzed using one-step RT-PCR kits (Clontech Laboratories, Inc., Palo Alto, Calif.). GS first-strand CDNA synthesis was performed from 1 μh of total RNA with 1×RT-AdvanTaq plus enzyme mix in 40 mM Tricine, 20 mM Kcl, 3 mM MgCl2, 3.75 ug/ml BSA, 0.2mM deoxynucleoside triphosphates, 400 pmol of oligo(dt) primer, and 200 pm GS primers (Sense 5′-GTGCTGACGTCTTTCTGGAG-3′, antisense 5′-CCAGCATCTTGTTCATGTCG-3′) in a final volume of 50 μl. The reaction mixtures were subjected to incubations for 60 min. at 50° C. in the Eppendorf Master gradient cycler (Westbury, N.Y). The reaction was stopped by heating at 95° C. for 5 min. Then PCR mixtures were subjected to 15 cycles of PCR amplification with a cycle profile including denaturation for 30 sec at 95° C., annealing for 30 sec at 65° C., and elongation for 2 min at 72° C., followed by 20 cycles of PCR amplification where a cycle was 1 min at 95° C., 1 min at 55° c., and 2 min at 72° C., respectively. Final extension was 5 min at 72° C. Human G3PDH primers were added to PCR reaction tubes at the same time as the internal control.

Analysis of RT-PCR Products: The amplification products of 10 μl of each PCR was separated in a 2% agarose gel, stained with 0.5 μg/ml ethidium bromide, and photographed. The band densities were evaluated (Alpha hnager 2000, San Leandro, Calif.). The absence of genomic DNA contamination was verified by control experiments that omit RNA or without reverse transcriptase in the RT step by preheating a master mix at 95° C. for 5 min to inactivate the enzyme.

Statistical Analysis: Statistical significance was evaluated using ANOVA. Comparison was made for insulin sensitivity and gene expression between CrPic and biotin alone and in combination when compared to control. Significance was accepted at the p<0.05 level.

Results

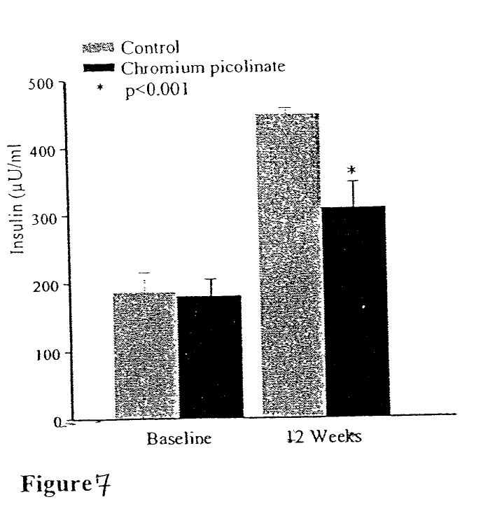

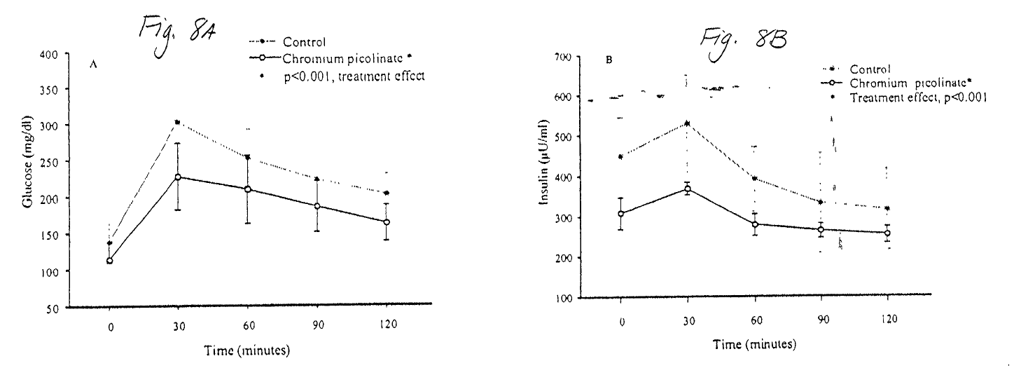

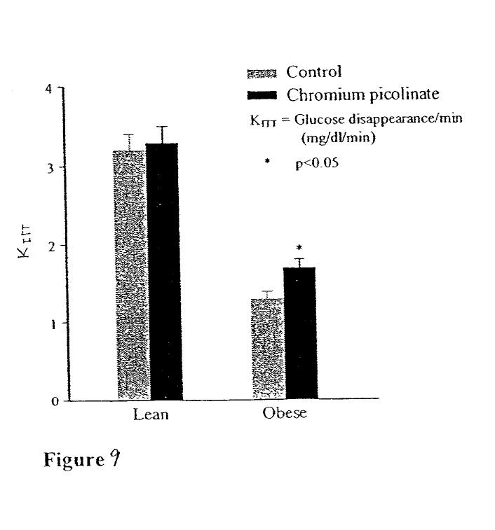

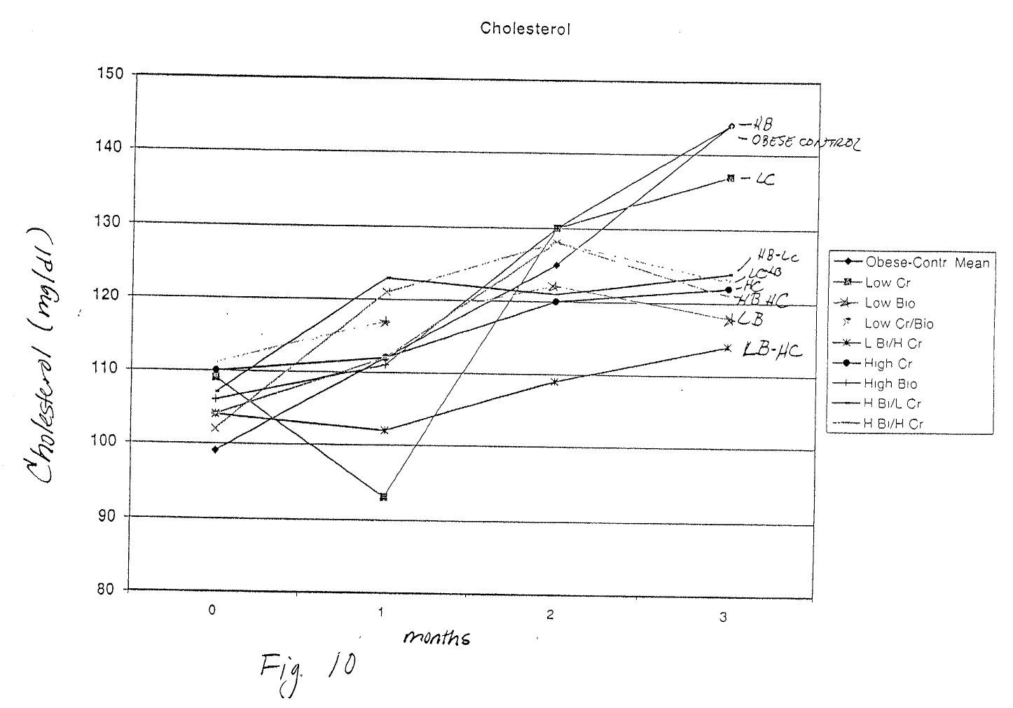

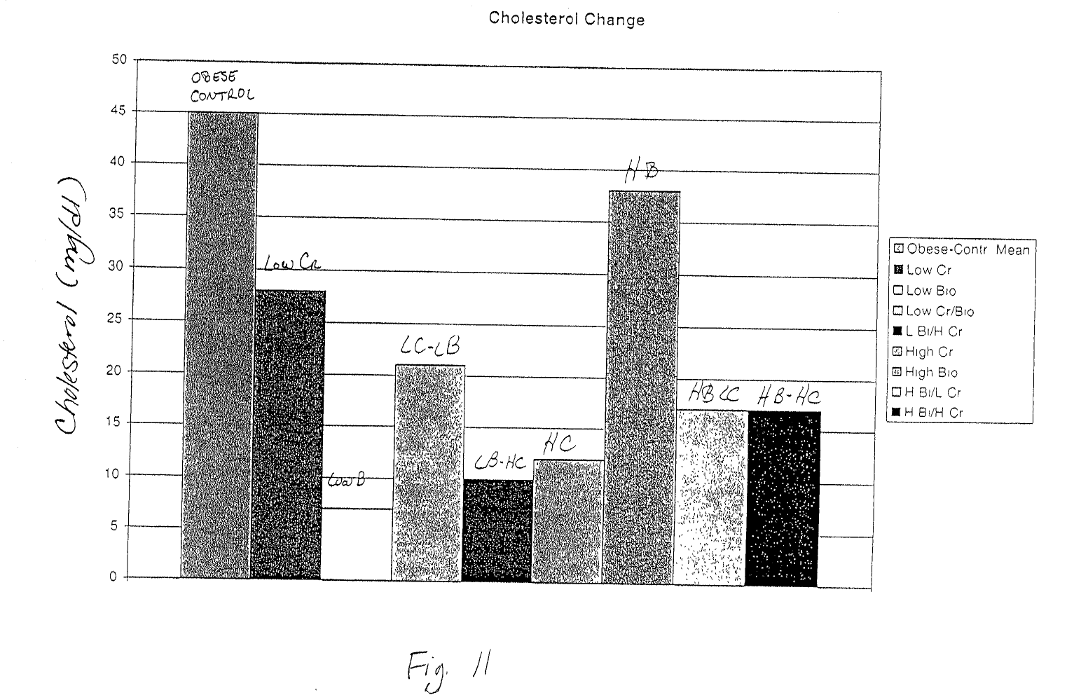

Our data strongly suggests that chromium may improve insulin signaling and we have demonstrated this in an animal model representative of the insulin resistance syndrome in vivo. Specifically, CrPic versus control solutions were given in hyperinsulinemic, insulin-resistant, obese JCR rats. The study was initiated on animals at approximately 4 months of age, and animals were treated specifically for three months. As demonstrated in FIG. 7, CrPic appeared to significantly lower fasting plasma insulin when assessed at the end of study. In addition, when comparing the glucose response to an intraperitoneal glucose tolerance test, CrPic significantly improved glucose response (see FIG. 8A), and significantly lowered insulin response in the obese rat (FIG. 8B). Insulin sensitivity, as assessed with an insulin tolerance test, was significantly improved with CrPic (FIG. 9). This was accomplished without any change in body weight in the animals. The improvement in insulin sensitivity was associated with a reduction in total cholesterol levels compared to lean animals (See, e.g., Table 2, and FIGS. 10 & 11). The effect of the combination of chromium and biotin on lipid levels in the animal study is detailed in Table 2. Specifically, the effect of chromium and biotin on cholesterol levels, high density lipid cholesterol (HDL-ch) levels, and triglyceride levels are recorded in Table 2.

TABLE 2
Chromium Picolinate + Biotin Animal Study
(Effect of Combination on Lipid Levels)

| | | | | |
|---|---|---|---|---|
| Month | 0 | 1 | 2 | 3 |

Cholesterol

| | | | | |
|---|---|---|---|---|
| Obese Control | 99 | 112 | 125 | 144 |
| Low Chromium | 109 | 93 | 130 | 137 |
| Low Biotin | 111 | 117 | 122 | 118 |
| Low Chromium/Low Biotin | 102 | 121 | 128 | 123 |
| Low Biotin/High Chromium | 104 | 102 | 109 | 114 |
| High Chromium | 110 | 112 | 120 | 122 |
| High Biotin | 106 | 111 | 130 | 144 |
| High Biotin/Low Chromium | 107 | 123 | 121 | 124 |
| High Biotin/High Chromium | 104 | 112 | 128 | 121 |
| Lean Control | 49 | 62 | 61 | 64 |

HDL-ch

| | | | | |
|---|---|---|---|---|
| Obese Control | 44.5 | 40.6 | 38.1 | 38.6 |
| Low Chromium | 45.9 | 38.6 | 43.4 | 45.9 |
| Low Biotin | 47.8 | 46.5 | 45.8 | 47.3 |
| Low Chromium/Low Biotin | 43 | 39.1 | 44.3 | 51.2 |
| Low Biotin/High Chromium | 44.9 | 43.7 | 48.9 | 51.3 |
| High Chromium | 43.1 | 43.2 | 51.5 | 48.1 |
| High Biotin | 43.6 | 42 | 46.8 | 46.9 |
| High Biotin/Low Chromium | 46.5 | 46 | 56 | 56.6 |
| High Biotin/High Chromium | 46.7 | 52.7 | 57.1 | 57.6 |
| Lean Control | 28.4 | 28.1 | 27.2 | 28.1 |

Triglyceride mg/dl

| | | | | |
|---|---|---|---|---|
| Obese Control | 318 | 293 | 356 | 362 |
| Low Chromium | 319 | 267 | 256 | 242 |
| Low Biotin | 317 | 331 | 272 | 229 |
| Low Chromium/Low Biotin | 286 | 296 | 207 | 206 |
| Low Biotin/High Chromium | 300 | 271 | 209 | 185 |
| High Chromium | 302 | 283 | 221 | 174 |
| High Biotin | 289 | 292 | 294 | 301 |
| High Biotin/Low Chromium | 298 | 336 | 321 | 352 |
| High Biotin/High Chromium | 296 | 254 | 240 | 161 |
| Lean Control | 73 | 73 | 73 | 68 |

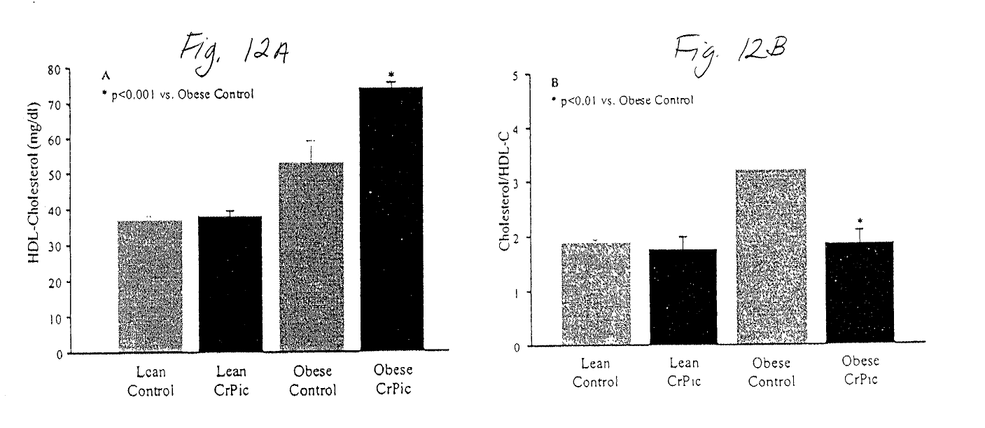

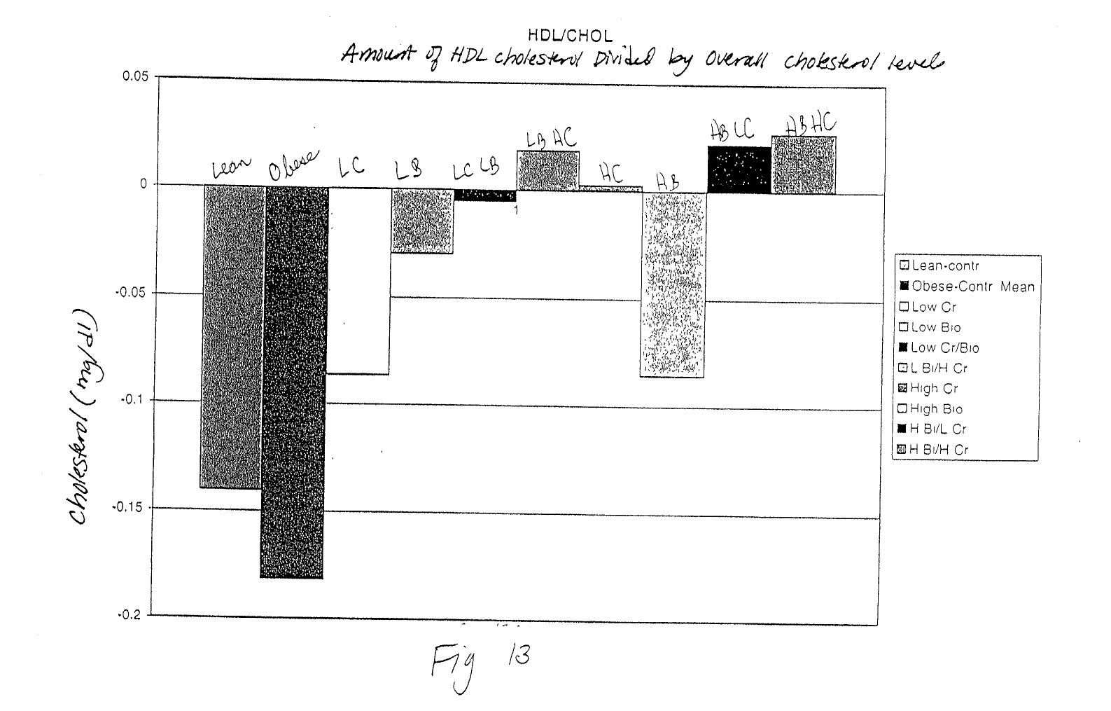

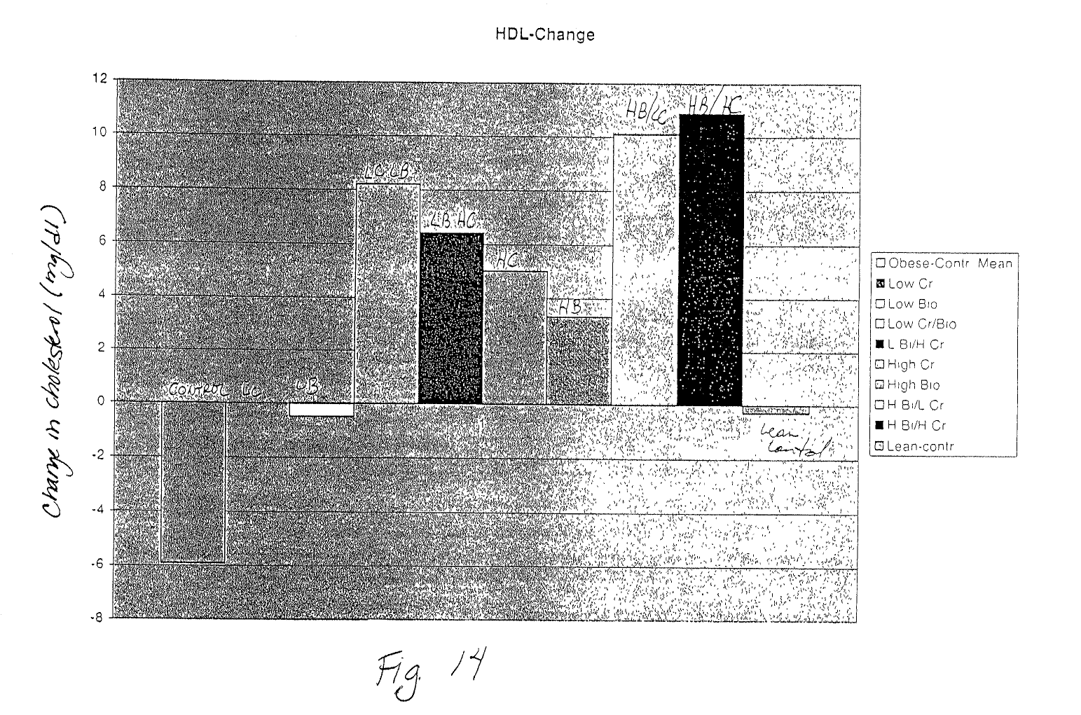

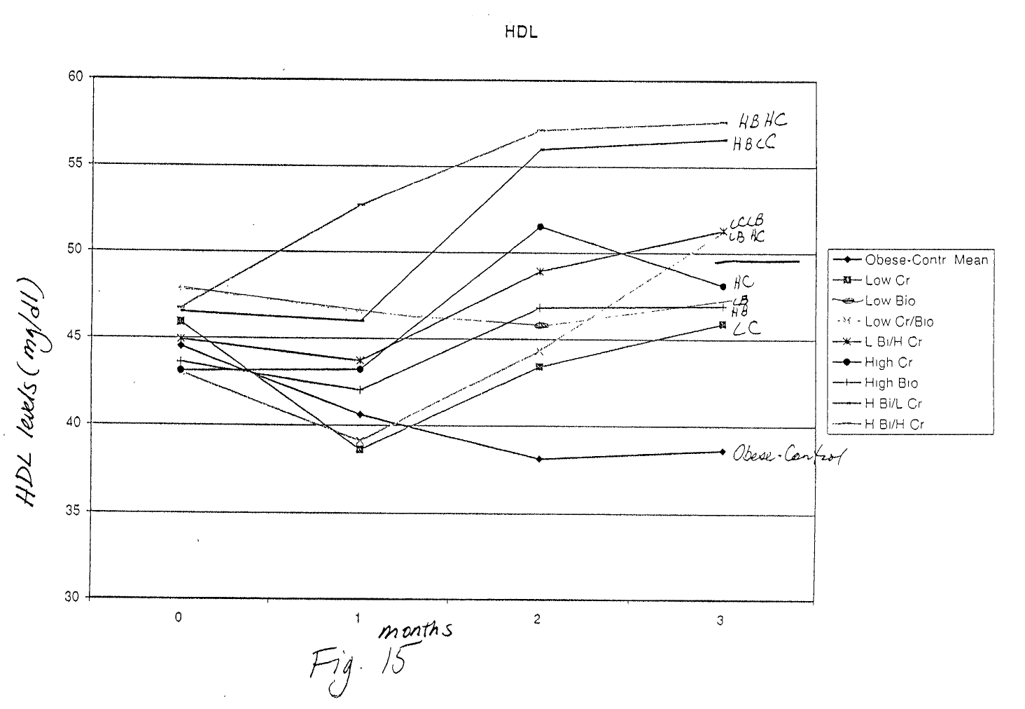

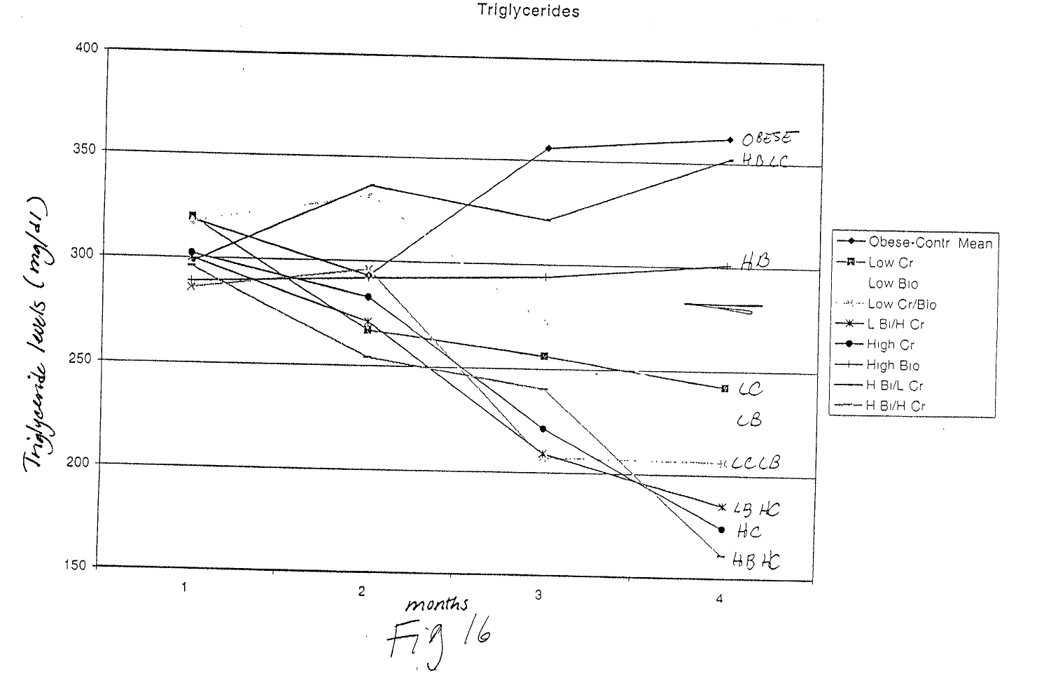

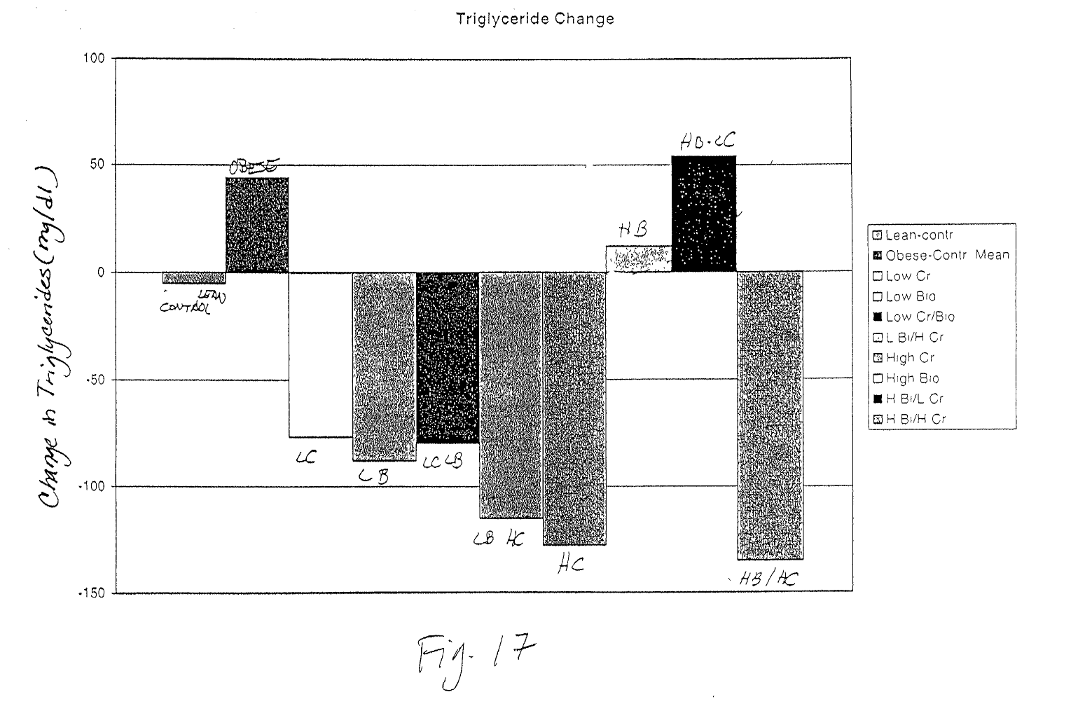

Further, the improvement in insulin sensitivity was associated with an improved HDL-C level (FIG. 12A) and a lowered cholesterol-HDL ratio (FIG. 12B). Finally, the co-administration of chromium and biotin resulted in the lowering of overall triglyceride portfolios in treated animals (FIGS. 16 and 17).

In summary, these animal studies strongly suggests that chromium and biotin have an in vivo effect to improve insulin resistance and components of the insulin resistance syndrome.

### Example 4

### Improved Blood Cholesterol Levels via Co-Administration of Chromium and Biotin

An individual presenting with high cholesterol is identified. Chromic tripicolinate and biotin are administered orally at a dose of 500 μg and 5 mg per day, respectively. An increase in HDL cholesterol and reduction of LDL cholesterol in the blood are observed over time.

### Example 5

### Attenuation in the Elevation in Blood Glucose Levels in People with Type 2 Diabetes via the Co-Administration of Chromium and Biotin

The effect of chromium and biotin on glycosylated hemoglobin levels in Type 2 diabetics was investigated. Specifically, a determination of the effects of chromium picolinate plus biotin incorporated into a carbohydrate containing beverage on blood glucose control in subjects with type 2 diabetes was initiated. Additionally, the effect of chromium picolinate and biotin on fasting blood glucose levels in type 2 diabetics ingesting a carbohydrate containing beverage was evaluated.

Study Design

The 12 week, randomized, double-blind, controlled clinical trial was conducted to determine the effects of chromium picolinate with biotin on blood glucose control in people with type 2 diabetes.

Thirty-four subjects were included in the evaluation (24 males, 10 females). Selection of the subjects was based on a number of criteria. Each subject presented with type 2 diabetes, had a fasting glucose level of greater than 90 but less than 170 mg/dL, and a glycosylated hemoglobin level greater than 7 mg/dL. A subject's hemoglobin level must have exceeded 12.5 g/dL and his or her BMI was greater than 26.0 but less than 39.0 kg/m². Baseline hypoglycemic medications remained stable throughout the treatment period.

The study included two parallel groups, a control group and a test group. Each group received beverages containing 29 g of carbohydrate, twice daily as a dietary supplement. The test and control beverages were similar in content to popular beverages designed for people with diabetes and included the following ingredients: water, maltodextrin, soy protein isolate, canola oil, inulin, cocoa powder, defatted, maltodextrin, crystalline fructose, vitamin and mineral mix, natural choclate flavor, lecithin, natural vanilla flavor, Nutrasweet® (NutraSweet Co., Deerfield, Ill., acesulfame K, and SeaKem (FMC Corp., Philadelphia, Pa.). The test group received beverages containing 300 μg chromium (as chromium picolinate) and 150 μg biotin. Table 3 details the nutritional breakdown of the beverage.

TABLE 3
Nutrient Beverage Composition

| Nutrients | With CrPic + Biotin | Control |
|---|---|---|
| Total Calories, KCal | 200 | 200 |
| Fat, g | 6.8 | 6.8 |
| Protein | 10.0 | 10.0 |
| Carbohydrates, g | 29.0 | 29.0 |
| Dietary Fiber, g | 5.4 | 5.4 |
| Chromium (as CrPic), μg | 300.0 | — |
| Biotin, μg | 150.0 | — |

N.B. Other vitamins and minerals were the same in both groups (based on % daily value and RDA); saturated fatty acids (SFA), monounsaturated fatty acids (MUFA), and polyunsaturated fatty acids (PUFA) are the same in each test group (source: Canola oil provides high MUFA and PUFA and low SFA).

The duration of the clinical trial was 12 weeks. At the start of the 12 week treatment, baseline data was gathered relating to glycosylated hemoglobin levels, fasting blood glucose concentrations, and fatigue. Indicators of blood glucose control were assessed at baseline, during the study, and at the end of the study. Specifically, subjects were evaluated at 0, 1, 2, 4, 6, 8, and 12 weeks.

Results

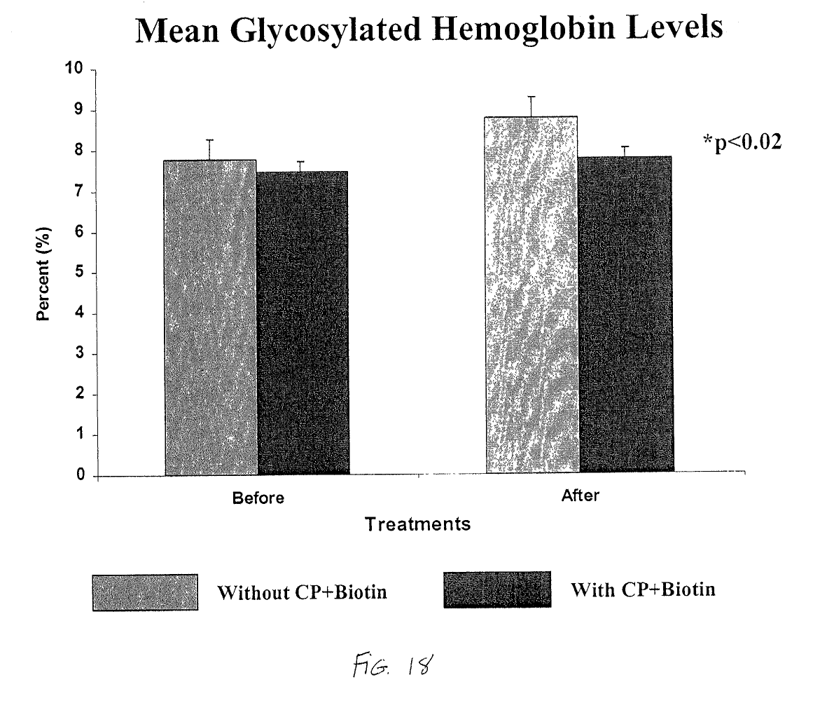

The effects of the co-administration of chromium and biotin on the attenuation in the elevation in blood glucose levels in people with type 2 diabetes who ingested a carbohydrate-containing beverage twice daily were studied. The results of the study are represented in FIGS. 18, 19, and 20. In FIG. 18, the change in mean glycosylated hemoglobin levels from baseline to the end of the week 12 is illustrated. A significant rise in glycosylated hemoglobin levels was observed in the control group given the carbohydrate containing beverage as compared to the treatment group administered chromium picolinate.

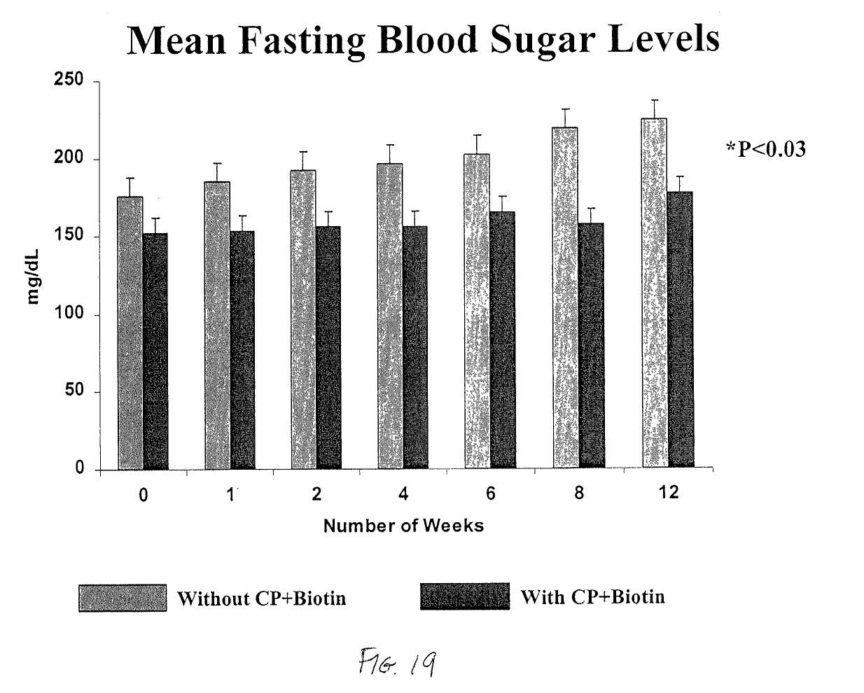

In FIG. 19, the change in mean fasting blood sugar levels (mg/dL) over the treatment period between subjects given chromium picolinate and biotin versus the control subjects is illustrated. Over time, the control group demonstrated an increase in mean fasting blood sugar levels over time as they continued to consume the carbohydrate-containing beverage. By contrast, subjects who consumed the carbohydrate-containing beverage supplemented with chromium picolinate and biotin did not exhibit a rise in mean fasting blood sugar levels.

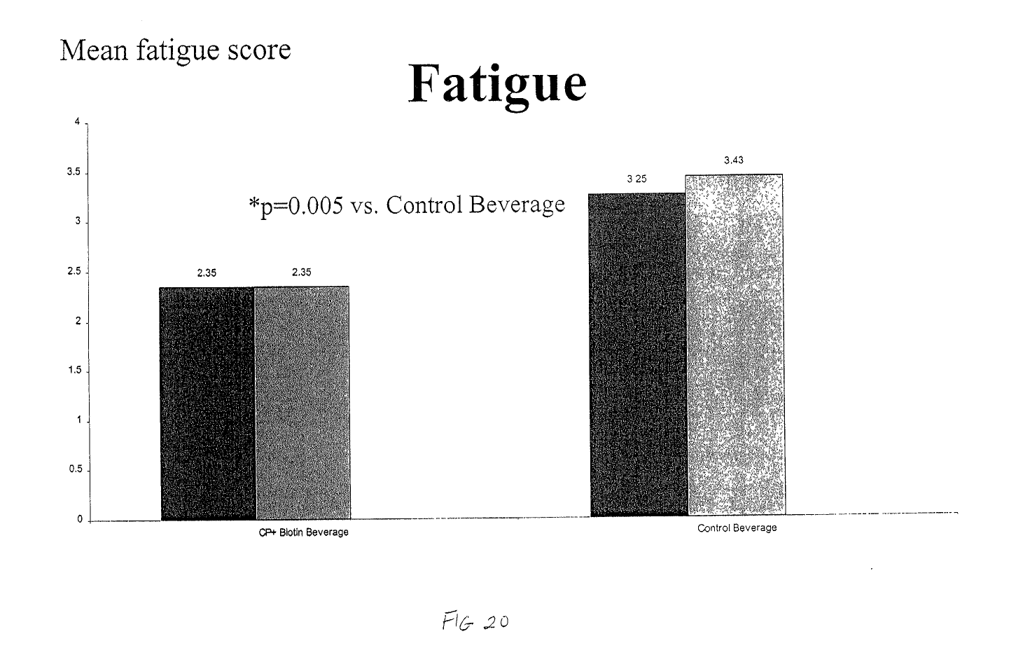

Changes in the subjects' energy levels were evaluated at the beginning of the study and after the end of the 12 week trial. FIG. 20 is a bar graph depicting differences in fatigue levels in subjects consuming a carbohydrate-containing beverage with chromium picolinate and biotin supplementation versus the control subjects, who consumed the carbohydrate-containing beverage without chromium picolinate and biotin. Fatigue was assessed using a 10-point Likert scale, wherein a score of 0 equaled no fatigue and a score of 10 equaled severe fatigue. Notably, no increase in fatigue was observed in the subjects treated with chromium picolinate and biotin.

Conclusions

Type 2 diabetic subjects administered a carbohydrate-containing beverage supplemented with chromium picolinate and biotin twice daily for twelve weeks demonstrated a reduced glycosylated hemoglobin level and lower blood glucose level than type 2 diabetic subjects who consumed the carbohydrate-containing beverage without the chromium picolinate and biotin. Without being limited to a particular theory, these phenomena may be based, in part, on the observations that chromium picolinate enhances insulin sensitivity by increasing the number of insulin receptors and/or by facilitating insulin binding at these receptors. Moreover, chromium potentiates the uptake of glucose in muscle cells and increases glycogen production. Biotin stimulates the activity of glucokinase in the liver, improves pancreatic islet cell function, and enhances insulin regulation of chromium III.

Our data has strongly suggested that chromium and biotin may improve post prandial hyperglycemia. As demonstrated in FIGS. 18 and 19, CrPic and biotin appeared to significantly lower blood glucose and glycated hemoglobin levels when assessed at the end of the study. This was accomplished without any change in fatigue of individuals with chromium picolinate and biotin beverage. In summary, these human studies indicate that CrPic and biotin may effect post prandial hyperglycemia. Moreover, the studies strongly suggest that the co-administration of a chromium complex in concert with biotin may reduce the glycemic index of food.

### Example 6

### The Co-Administration of a Chromium Complex for the Reduction in the Glycemic Index of a Fruit Juice

A chromium complex plus biotin act to lower the glycemic index of orange juice. 600 μg of chromium histidinate and 300 ig of biotin are formulated as a liquid and added to eight ounces of orange juice. The chromium histidinate and biotin are mixed with the orange juice and consumed by an individual. A 10-25 mg/dL reduction of blood sugar is observed as compared to the blood sugar of an individual consuming orange juice which has not be supplemented with chromium histidinate and biotin. The glycemic index of the orange juice is lowered.

### Example 7

### The Co-Administration of a Chromium Complex for the Reduction in the Glycemic Index of a Fructose-Sweetened Carbonated Cola-Flavored Beverage

A chromium complex plus biotin act to lower the glycemic index of a fructose-sweetened carbonated cola-flavored beverage. 600 μg of chromium picolinate and 300 μg of biotin are formulated as a liquid and added to eight ounces of the beverage. The chromium picolinate and biotin are mixed with the beverage and consumed by an individual. A 10-25 mg/dL reduction of blood sugar is observed as compared to the blood sugar of an individual consuming fructose-sweetened carbonated cola-flavored beverage which has not be supplemented with chromium picolinate and biotin. The glycemic index of the fructose-sweetened carbonated cola-flavored beverage is lowered.

### Example 8

### Chromium Polynicotinate and Biotin for the Reduction in the Glycemic Index of a High-Carbohydrate Food

A compound comprising 300 μg chromium polynicotinate and 200 μg biotin is formulated as a powder and sprinkled on top of prepared pasta. The pasta is consumed by an individual. Shortly after the pasta has been consumed, a sample of the individual's blood is tested for glycosylated hemoglobin and blood sugar levels. A reduction in the individual's glycosylated hemoglobin and blood sugar levels is observed. The co-administration of chromium polynicotinate and biotin acts to lower the glycemic index of the pasta, thereby minimizing the sharp elevation in glucose response one would expect to observe when an individual consumes pasta without the chromium polynicotinate and biotin supplementation.

The above description of the invention is set forth solely to assist in understanding the invention. It is to be understood that variations of the invention, including all equivalents now known or later developed, are to be considered as falling within the scope of the invention, which is limited only by the following claims.

What is claimed is:

1. A method for treating dyslipidemia comprising administering to an individual in need thereof an effective dose of a chromium complex and biotin.

2. The method of claim 1, wherein the effective dose of chromium complex is between about 25 and 2,000 micrograms per day.

3. The method of claim 1, wherein the effective dose of chromium complex is between about 300 and 1,000 micrograms per day.

4. The method of claim 1, wherein the effective dose of biotin is between about 25 μg and 20 mg per day.

5. The method of claim 1, wherein the effective dose of biotin is between about 150 μg and 5 mg.

6. The method of claim 1, wherein said dyslipidemia is caused by elevated levels of LDL cholesterol in the blood.

7. The method of claim 1, wherein said dyslipidemia is caused by low levels of HDL cholesterol in the blood.

8. The method of claim 1, wherein said dyslipidemia is caused by elevated levels of triglyceride in the blood.

9. The method of claim 1, wherein said chromium complex is selected from the group consisting of chromium picolinate, chromic tripicolinate, chromium nicotinate, chromic polynicotinate, chromium chloride, chromium histidinate, and chromium yeasts.

10. The method of claim 1, wherein said chromium complex is in a pharmaceutically acceptable carrier.

11. The method of claim 1, wherein said biotin is in a pharmaceutically acceptable carrier.

12. The method of claim 1, wherein said chromium complex is orally administered.

13. The method of claim 1, wherein said biotin is orally administered.

14. The method of claim 1, wherein said chromium complex is parenterally administered.

15. The method of claim 1, wherein said biotin is parenterally administered.

16. The method of claim 1, further comprising administering picolinic acid.

17. The method of claim 1, further comprising administering nicotinic acid.

18. The method of claim 16, further comprising administering nicotinic acid.

19. The method of claim 1, wherein said chromium complex and said biotin are administered simultaneously.

20. The method of claim 1, wherein said biotin is administered within 24 hours of said chromium complex.

21. A composition consisting essentially of a chromium complex and biotin, wherein the ratio of chromium complex to biotin is from about 1:1,000 to about 100:1 (w/w).

22. The composition of claim 21, wherein said chromium complex is selected from the group consisting of chromium picolinate, chromic tripicolinate, chromium nicotinate, chromic polynicotinate, chromium chloride, chromium histidinate, and chromium yeasts.

23. A method of reducing the glycemic index of food comprising administering to said food an effective amount of a chromium complex and biotin.

24. The method of claim 23, wherein said chromium complex is selected from the group consisting of chromium picolinate, chromic tripicolinate, chromium nicotinate, chromic polynicotinate, chromium chloride, chromium histidinate, and chromium yeasts.

25. The method of claim 23, wherein between about 50 μg and 750 μg of said chromium complex is administered to the food.

26. The method of claim 23, wherein between about 50 μg and 1 g of biotin are administered to the food.

27. The method of claim 23, wherein said chromium complex and said biotin are administered simultaneously.

28. The method of claim 23, wherein said chromium complex is added within one hour of said biotin complex.

29. The method of claim 23, wherein said chromium complex and said biotin are administered as a powder, liquid, oil suspension, granule, emulsion, syrup, elixir, or beverage.

30. A food having a reduced glycemic index prepared by the method of claim 23.

31. A method for lowering post prandial hyperglycemia comprising administering to a subject in need thereof an effective amount of a chromium complex and biotin.

32. The method of claim 31, wherein said chromium complex is selected from the group consisting of chromium picolinate, chromic tripicolinate, chromium nicotinate, chromic polynicotinate, chromium chloride, chromium histidinate, and chromium yeasts.

33. The method of claim 31, wherein said subject is administered between about 25 and 2,000 micrograms per day of a chromium complex and between about 25 μg and 20 mg per day of biotin.

34. The method of claim 31, comprising administering between about 300 and 1,000 micrograms per day of a chromium complex.

35. The method of claim 31, comprising administering between about 150 μg and 5 mg biotin per day.

36. The method of claim 31, wherein said chromium complex and said biotin are administered simultaneously.

37. The method of claim 31, wherein said biotin is administered within 24 hours of said chromium complex.
